(19) United States
(12) Patent Application Publication
(10) Pub. No.: US 2020/0225249 A1
(43) Pub. Date: Jul. 16, 2020

(54) COMPOSITIONS AND METHODS FOR THE DIAGNOSIS AND TREATMENT OF DISEASES OF THE LIVER

(71) Applicant: Viscient Biosciences, Inc., San Diego, CA (US)

(72) Inventors: Jeffrey N. Miner, San Diego, CA (US); Traci Ostertag, San Diego, CA (US); Phil Tan, San Diego, CA (US); Ping Jin, San Diego, CA (US)

(21) Appl. No.: 16/738,265

(22) Filed: Jan. 9, 2020

Related U.S. Application Data
(60) Provisional application No. 62/791,658, filed on Jan. 11, 2019.

(51) Int. Cl. G01N 33/68

(52) U.S. Cl. CPC G01N 33/6893; G01N 2500/04

(57) ABSTRACT

Provided herein are novel molecular markers and targets of liver disease, including NAFLD, NASH, liver fibrosis and related conditions. Also provided herein are methods of screening for modulators of such molecular markers and targets for the treatment of diseases of the liver as well as the modulators useful for treating such disease. Also provided are novel molecular markers useful for diagnosing diseases of the liver, including, NAFLD, NASH, liver fibrosis and related conditions, and for monitoring the progression and treatment of such disease of the liver.

### CROSS-REFERENCE TO RELATED APPLICATIONS

This present application claims priority to U.S. Provisional Patent Application Ser. No. 62/791,658 filed Jan. 11, 2019, the disclosure of which is incorporated herein by reference in its entirety.

### SEQUENCE LISTING

The instant application contains a Sequence Listing which was originally submitted electronically on Jan. 9, 2020. A Corrected Sequence Listing to overcome certain deficiencies in ASCII format and submitted electronically on Jan. 31, 2020 is hereby incorporated by reference in its entirety. Said Corrected ASCII copy, created on Jan. 17, 2020, is named 1428-2001US-ST25 and is 58,827 bytes in size.

### FIELD OF THE INVENTION

The invention relates generally to diseases of the liver, and more specifically to novel molecular markers and targets and therapeutic compositions and methods for the diagnosis and treatment of liver diseases. Furthermore, the invention relates to methods of screening for therapeutic compounds and related treatments for liver disease.

### BACKGROUND

One of the greatest challenges in modern medicine is the accurate diagnosis, monitoring and treatment of disease. Recent advances in the area of genetics and cellular biology have led to the discovery that many seemingly identical diseases may have very different underlying causes of action at a genetic level. As such, even if a clinician can accurately identify the symptoms of a disease and the tissues that are affected by the disease, it is often difficult to select an appropriate treatment.

Once such area of grave concern to the world is the development of metabolic syndromes and related pathologies. These include numerous diseases of the liver, such as steatohepatitis (a fatty liver disease commonly seen in chronic alcoholics) and nonalcoholic fatty liver disease (NAFLD). Due to the increased incidence of obesity in the United States, as many as 30% of adult Americans may be afflicted with NAFLD, which is characterized by hepatocyte lipid droplet buildup. In a small subset of NAFLD patients, the disease will progress to a much more severe disorder known as nonalcoholic steatohepatitis (NASH). NASH is characterized by inflammation, fibrosis, and damage to the liver tissues, and if left untreated will often progress to liver cirrhosis and eventual loss of liver function followed by transplantation and/or death.

While NAFLD/NASH is relatively easy to diagnose due to the gross anatomical symptoms associated with the disease, the precise cellular mechanisms underlying the disease have remained elusive. This problem is made even more difficult to solve by the presence of numerous cell types within the liver tissue, each of which may contribute to different aspects of disease progression. While certain treatments for NASH have been identified (see Abdul Oseini, Therapies In Non-Alcoholic Steatohepatitis (Nash), Liver Int. 2017 January; 37(Suppl 1): 97-103), the diagnostic uncertainty discussed above often renders it difficult to select the appropriate treatment. As such, there is a need for a method of identifying the underlying molecular components that play a role in the development and progression of NAFLD and NASH. Such components may serve a number of beneficial roles, including serving as markers and targets for pharmaceutical intervention, screening for changes in the component's activity as a method of diagnosis, or by intentionally altering the component's expression to learn more about liver disease progression and to screen for and discover effective novel pharmaceutical compounds and treatments for NAFLD, NASH, liver fibrosis and related conditions.

### SUMMARY OF THE INVENTION

The above identified shortcomings of the prior art are addressed by the present invention. Presented herein are unique molecular markers of liver disease, including NAFLD, NASH, liver fibrosis and related conditions previously unknown in the diagnosis and treatment of liver disease. In addition, further presented herein are methods of screening for modulators useful to treat liver disease as well as the specific modulators of the present unique molecular marker targets capable or treating and or halting the progression of such liver diseases. Also provided are methods of using said molecular markers to diagnose patients susceptible to such liver diseases and to monitor their treatment and disease progression.

Thus, it is one aspect of the present invention to present a method of screening diseased tissues to identify novel molecular markers which are differentially expressed in such diseased liver tissues relative to healthy tissues of the same type. In certain embodiments of the present invention the tissues will be liver tissues. Preferably, such liver tissues will be separated into individual cell types, including hepatocytes, endothelial cells (ECs), hepatic stellate cells (hSCs) and Kupffer cells, prior to analysis. In certain embodiments, such individual cell types may be analyzed individually, or in specific combinations of cell types and can preferably include bioprinted tissues, sphereroid tissue culture or whole human liver tissue in certain embodiments, such cell types may comprise endothelial cells (ECs), hepatic stellate cells (hSCs), and Kupffer cells. In certain embodiments, the disease being assayed will be NAFLD/ NASH as well as fibrosis of the liver. Preferably, the screen will employ certain statistical analyses, described herein, to identify the specific targets which are differentially expressed in the diseased tissues relative to those of a healthy patient.

In another aspect of the present invention previously unknown and novel molecular markers of liver diseases are provided including LOXL4, CHRM2, DMKN, QPRT and SLC12A8. Preferably, the molecular marker proteins comprise SEQ. ID NO. 1 through SEQ ID NO. 21 and amino acid sequences that are at least about 70% similar, preferably at least about 80% similar, more preferably at least about 90% similar and most preferably at least about 95% similar (e.g., 95%, 96%, 97% , 98%, 99%, 100%) to any of the molecular markers at SEQ ID NO. 1 through SEQ ID NO. 21. Each of these novel markers show many fold increased expression in diseased liver tissue relative to healthy liver tissue, both at the single cell, multicell and bioprinted tissue levels. Preferably such molecular markers are used as diagnostic tools to enhance the identification, progression, monitoring and treatment of patients with liver diseases, including, but not limited to, NAFLD, NASH, liver fibrosis and related conditions.

It is another aspect of the present invention to provide a list of molecular markers that also serve as molecular targets which are differentially expressed in liver tissue of patients suffering from liver disease, including NAFLD and NASH, relative to healthy liver tissue and are useful in therapeutic intervention in liver disease. Accordingly, such molecular targets play a regulatory or modulatory role in liver disease and its progression. More preferably these molecular targets include LOXL4, CHRM2, DMKN, QPRT and SLC12A8. Preferably, the molecular target proteins comprise SEQ. ID NO. 1 through SEQ ID NO. 21 and amino acid sequences that are at least about 70% similar, preferably at least about 80% similar, more preferably at least about 90% similar and most preferably at least about 95% similar (e.g., 95%, 96%, 97%, 98%, 99%, 100%) to any of the molecular markers at SEQ ID NO. 1 through SEQ ID NO. 21. In certain embodiments of the present invention, the activity of such molecular targets may be modulated by administration of one or more modulator compounds in order to provide therapeutic efficacy to a patient. Preferably, such a patient will be exhibiting any symptoms of liver disease, and more preferably, such a patient will be diagnosed with NAFLD, NASH, liver fibrosis and/or related conditions. In certain other embodiments of the present invention, such molecular targets are useful to screen for novel modulators of liver disease.

In yet other embodiments, the activity of such molecular targets may be intentionally modulated in order to create experimental models of liver disease. In certain embodiments, such targets may be modulated in individual cells, in two-dimensional cell cultures, in three-dimensional cell cultures including microspheres, in bio-printed tissue aggregates, in whole tissue, and in whole-organism based experimental models. The activity state of such tissues may be modulated uniformly, in individual cell types within a greater aggregate, or in individual cells within such an aggregate. Modulation of such activity may be done using any method currently known in the art or later developed.

In still another embodiment of the present invention, the activity of such molecular targets may be modulated in a preexisting or novel experimental model of liver disease (including those disclosed in Maddalena Parafati, A nonalcoholic fatty liver disease model in human induced pluripotent stem cell-derived hepatocytes, created by endoplasmic reticulum stress-induced steatosis, Disease Models & Mechanisms 2018 11: dmm033530 doi: 10.1242/dmm.033530) in order to assess the effect of such modulation on the state of the disease in such an experimental model of liver disease. In certain embodiments, the modulation may slow the progression of disease, may arrest the progression of the disease, may improve the health of the experimental model of disease, or may result in the experimental model of disease resembling a completely healthy state.

Thus it is one embodiment of the present invention to present a method of treating diseases of the liver in a patient, the method comprising: identifying a patient in need of treatment; and modulating the activity state of one or more molecular targets of the present invention associated with liver disease in said patient; wherein said molecular targets associated with liver disease comprise targets selected from a list consisting of a least one of LOXL4, CHRM2, DMKN, QPRT and SLC12A8. Preferably the modulators comprise natural or synthetic modulators selected from cytokines, cytokine variants, analogues, muteins, antibodies, binding compounds derived from antibodies, small molecules, peptide mimetics, siRNA, nucleic acids, proteins or an extract made from biological materials such as bacteria, plants, fungi, or animal cells or tissues. Preferably such methods and modulators are also useful to prevent said diseases of the liver.

In yet another embodiment of the present invention, novel modulators of liver disease discovered according to the methods of the present invention are provided. Such modulators can be natural or synthetic compounds or constructs. Preferably, such modulators comprise cytokines, cytokine variants, analogues, muteins, antibodies, binding compounds derived from antibodies, small molecules, peptide mimetics, siRNA, nucleic acids, proteins or an extract made from biological materials such as bacteria, plants, fungi, or animal cells or tissues. In a preferred embodiment the modulators modulate the activity and/or expression of the molecular targets, including LOXL4, CHRM2, DMKN, QPRT and SLC12A8. In one embodiment, the modulators inhibit the activity and/or expression of the molecular targets and in an alternative embodiment, the molecular targets enhance or increase the expression and/or activity of the molecular targets. In an especially preferred embodiment, the modulator comprises an activator of QPRT.

These, and other, embodiments of the invention will be better appreciated and understood when considered in conjunction with the following description and the accompanying tables. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

### DETAILED DESCRIPTION OF THE INVENTION

Before the invention is described in detail, it is to be understood that this invention is not limited to the particular component parts or structural features of the molecular markers, molecular targets, modulators, compositions described or process steps of the methods described as such markers, targets, modulators, compositions and methods may vary. It is also to be understood that the terminology used herein is for purposes of describing particular embodiments only and is not intended to be limiting. Accordingly, in addition to any definitions provided herein, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described.

The mere fact that certain measures are recited in mutually different dependent claims does not indicate that a combination of these measures cannot be used to advantage. Any reference signs in the claims should not be construed as limiting the scope. It must be noted that, as used in the specification and the appended claims, the singular forms “a,” “an” and “the” include singular and/or plural referents unless the context clearly dictates otherwise. Further, in the claims, the word “comprising” does not exclude other elements or steps.

It is moreover to be understood that, in case parameter ranges are given which are delimited by numeric values, the ranges are deemed to include these limitation values.

All of the patents and publications referred to herein are incorporated by reference in their entirety.

### Definitions:

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, “nonalcoholic fatty liver disease” or “NAFLD” refers to a condition in which fat is deposited in the liver (hepatic steatosis), with or without inflammation and liver fibrosis, in the absence of excessive alcohol use.

As used herein, “steatosis” and “non-alcoholic steatosis” are used interchangeably, and include mild, moderate, and severe steatosis, without inflammation or liver fibrosis, in the absence of excessive alcohol use.

As used herein, “nonalcoholic steatohepatitis” or “NASH” refers to NAFLD in which there is inflammation and/or fibrosis in the liver. NASH may be divided into four stages. Exemplary methods of determining the stage of NASH are described, for example, in Kleiner et al., 2005, Hepatology, 41(6):1313-1321, and Brunt et al., 2007, Modern Pathol., 20: S40-S48.

As used herein, “liver fibrosis” refers to the excessive accumulation of extracellular matrix proteins including collagen that occurs in most types of chronic liver diseases. (Bataller & Brenner. Liver fibrosis. J Clin Invest 2005; 115:209-18), including hepatitis B virus (HBV), hepatitis C virus (HCV), alcoholic liver disease and non-alcoholic steatohepatitis (NASH) (Bataller & Brenner 2005). Liver fibrosis is also manifested by scar formation. “Cirrhosis” is a late stage of hepatic fibrosis that has resulted in widespread distortion of normal hepatic architecture. Cirrhosis is characterized by regenerative nodules surrounded by dense fibrotic tissue. Symptoms may not develop for years and are often nonspecific (e.g., anorexia, fatigue, weight loss). Late manifestations include portal hypertension, ascites, and, when decompensation occurs, liver failure. Diagnosis often requires liver biopsy. Cirrhosis is usually considered irreversible.

As used herein, reference herein to “normal cells” or “healthy cells or tissue” means cells and tissues that are from the same organ and of the same type as the cells or tissues exhibiting liver disease. In one aspect, the corresponding normal cells comprise a sample of cells obtained from a healthy individual. Such corresponding normal cells can, but need not be, from an individual that is age-matched and/or of the same sex as the individual providing the diseased cells being examined. In another aspect, the corresponding normal cells comprise a sample of cells obtained from an otherwise healthy portion of tissue of a subject having liver disease.

The term “molecular marker” refers a DNA, related nucleic acids (e.g., RNA, mRNA etc.) and protein(s) transcribed from said DNA that is an indicator of disease and are capable of being used as a marker of disease (e.g., liver disease, including NAFLD, NASH, liver fibrosis and related conditions), including disease onset, progression, monitoring, diagnosis and treatment. Accordingly, a molecular marker is an anatomic, physiologic, biochemical, or molecular parameter associated with the presence of a specific physiological state or process (e.g. disease or condition), whether normal or abnormal, and, if abnormal, whether chronic or acute. Molecular markers are detectable and measurable by a variety of methods including laboratory assays and medical imaging. A molecular marker may be differentially present at any level, but is generally present at a level that is increased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more; or is generally present at a level that is decreased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent).

The term “molecular target” is a subset of molecular markers and refers to a DNA, related nucleic acids (e.g., RNA, mRNA etc.) and protein(s) transcribed from said DNA that are an integral part of the disease pathway in the liver and that are capable of being modulated by a potential drug or therapeutic compound to discover, design, develop and deploy such drug or therapeutic compound to treat said liver disease, including, but not limited to NAFLD, NASH, liver fibrosis and related conditions.

The term “modulator” refers to a chemical compound (naturally occurring or synthesized), such as a biological macromolecule (e.g., nucleic acid, protein, non-peptide, or organic molecule), or an extract made from biological materials such as bacteria, plants, fungi, or animal (particularly mammalian) cells or tissues, or even an inorganic element or molecule. Accordingly, the modulator can be virtually any chemical compound. It can exist as a single isolated compound or can be a member of a chemical (e.g., combinatorial) library. In one embodiment, the modulator is a small organic molecule. The term small organic molecules refers to molecules of a size comparable to those organic molecules generally used in pharmaceuticals. The term excludes biological macromolecules (e.g., proteins, nucleic acids, etc.). In certain embodiments, small organic molecules range in size up to about 5000 Da, up to 2000 Da, or up to about 1000 Da. The compound may be an antibody.

“Activity” of a molecule, including a Modulator, may describe or refer to the binding of the molecule to a ligand or to a receptor, to catalytic activity; to the ability to stimulate gene expression or cell signaling, differentiation, or maturation; to antigenic activity, to the modulation of activities of other molecules, and the like. “Activity” of a molecule such as a Modulator may also refer to activity in modulating or maintaining cell-to-cell interactions, e.g., adhesion, or activity in maintaining a structure of a cell, e.g., cell membranes or cytoskeleton. “Activity” can also mean specific activity, e.g., [catalytic activity]/[mg protein], or [immunological activity]/[mg protein], concentration in a biological compartment, or the like. “Activity” may refer to modulation of components of the innate or the adaptive immune systems.

“Modulating,” “Modulation,” “Activation,” “stimulation,” “inhibition,” and “treatment,” as it applies to cells or to receptors, may have the same meaning, e.g., modulating, modulation, activation, stimulation, inhibition or treatment of a cell or receptor with a ligand, unless indicated otherwise by the context or explicitly. “Ligand” encompasses natural and synthetic ligands, e.g., cytokines, cytokine variants, analogues, muteins, and binding compounds derived from antibodies. “Ligand” also encompasses small molecules, e.g., peptide mimetics of cytokines and peptide mimetics of antibodies. Activation, modulation and inhibition can refer to cell activation, modulation or inhibition as regulated by internal mechanisms as well as by external or environmental factors. “Response,” e.g., of a cell, tissue, organ, or organism, encompasses a change in biochemical or physiological behavior, e.g., concentration, density, adhesion, or migration within a biological compartment, rate of gene expression, or state of differentiation, where the change is correlated with modulation, modulation, activation, stimulation, inhibition or treatment, or with internal mechanisms such as genetic programming.

“Administration” and “treatment,” as it applies to an animal, human, experimental subject, cell tissue, organ, or biological fluid, refers to contact of an exogenous pharmaceutical, therapeutic, diagnostic agent, modulator or composition to the animal, human, subject, cell, tissue, organ, or biological fluid. “Administration” and “treatment” can refer, e.g., to therapeutic, pharmacokinetic, diagnostic, research, and experimental methods. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell. The term “subject” includes any organism, preferably an animal, more preferably a mammal (e.g., rat, mouse, dog, cat, rabbit) and most preferably a human, including a human patient.

“Treat” or “treating” refers to administering a therapeutic agent, such as a composition containing any of the liver disease modulators or similar compositions described herein, internally or externally to a subject or patient having one or more disease symptoms, or being suspected of having a disease or being at elevated at risk of acquiring a disease, for which the agent has therapeutic activity. Gene editing technology such as CRISPR/cas9 methods may also be utilized to carry out liver specific reduction of molecular markers or molecular targets and/or related co-factors.

The activity of a molecular marker or molecular target may be “modulated” in numerous ways. By way of example and not limitation the activity of a molecular marker or molecular target may be modulated by administering an exogenous agent, including a modulator, that directly interacts with the molecular marker or molecular target gene or gene product. Alternately, a modulator may be administered which interacts with components that exist in a pathway either upstream or downstream of the molecular marker or molecular target in order to achieve the desired result. By way of example, if the molecular target is a receptor, a modulator may be administered which directly binds to the receptor, or which binds to a ligand in order to prevent the ligand from interacting with the molecular target receptor. In yet another alternative, a modulator may be administered which prevents the expression of the gene which encodes the molecular target receptor. In certain embodiments, the modulator may be administered in combination with a pharmaceutical excipient or carrier.

Typically, the modulator is administered in an amount effective to alleviate one or more disease symptoms in the treated subject or population, whether by inducing the regression of or inhibiting the progression of such symptom(s) by any clinically measurable degree. The amount of a therapeutic agent that is effective to alleviate any particular disease symptom (also referred to as the “therapeutically effective amount”) may vary according to factors such as the disease state age, and weight of the patient, and the ability of the drug to elicit a desired response in the subject Whether a disease symptom has been alleviated can be assessed by any clinical measurement typically used by physicians or other skilled healthcare providers to assess the severity or progression status of that symptom. While an embodiment of the present invention (e.g., a treatment method or article of manufacture) may not be effective in alleviating the target disease symptom(s) in every subject, it should alleviate the target disease symptom(s) in a statistically significant number of subjects as determined by any statistical test known in the art such as the Student's t-test, the chi squared-test, the U-test according to Mann and Whitney, the Kruskal-Wallis test (H-test), Jonekheere-Terpstra-test and the Wilcoxon-test.

Furthermore, the molecular marker or molecular target may be modulated genetically. Genetic modifications include genome editing techniques, including so-called knock-in therapies, wherein one version of a gene is substituted for another version of the same gene and knock-out therapies, wherein the target gene is rendered non-functional. See, e.g. Matthew Porteus, Genome Editing: A New Approach to Human Therapeutics, Annual Review of Pharmacology and Toxicology, 2016 56:1, 163-190; Morgan L Maeder and Charles A Gersbach, Genome-editing Technologies for Gene and Cell Therapy, Mol Ther. 2016 March; 24(3): 430-446; Sergiu Chira, et. al. CRISPR/Cas9: Transcending the Reality of Genome Editing, Nucleic Acids, Jun. 16, 2017, VOLUME 7, P211-222. Such modulation also includes so-called knock-down therapies, wherein the gene activity is modulated by preventing mRNA translation. Such techniques may make use of microRNA, siRNA, shRNA, or other methods of inhibiting mRNA translation, including the use of alternate antisense oligonucleotides. In certain embodiments, a gene may be knocked down in order to provide therapeutic efficacy to a patient. In other embodiments, the knock-down of the endogenous gene may be accompanied by replacement with a therapeutically efficacious version of the gene. See, e.g., F P Manfredsson, et al., RNA knockdown as a potential therapeutic strategy in Parkinson's disease, Gene Therapy volume 13, pages517-524 (2006); Artur V. Cideciyan, et al., Mutation-independent rhodopsin gene therapy by knockdown and replacement with a single AAV vector, PNAS Sep. 4, 2018, 115 (36) E8547-E8556.

“Diagnose”, “diagnosing”, “diagnosis”, and variations thereof refer to the detection, determination, or recognition of a health status or condition of an individual on the basis of one or more signs, symptoms, data, or other information pertaining to that individual. The health status of an individual can be diagnosed as healthy/normal (i.e., a diagnosis of the absence of a disease or condition) or diagnosed as ill/abnormal (i.e., a diagnosis of the presence, or an assessment of the characteristics, of a disease or condition). The terms “diagnose”, “diagnosing”, “diagnosis”, etc., encompass, with respect to a particular disease or condition, the initial detection of the disease; the characterization or classification of the disease; the detection of the progression, remission, or recurrence of the disease; and the detection of disease response after the administration of a treatment or therapy to the individual. The diagnosis of NAFLD includes distinguishing individuals who have NAFLD from individuals who do not. The diagnosis of NASH includes distinguishing individuals who have NASH from individuals who have steatosis in the liver, but not NASH, and from individuals with no liver disease.

“Prognose”, “prognosing”, “prognosis”, and variations thereof refer to the prediction of a future course of a disease or condition in an individual who has the disease or condition (e.g., predicting patient survival), and such terms encompass the evaluation of disease response after the administration of a treatment or therapy to the individual.

As used herein, a “therapeutically effective” treatment refers to a treatment that is capable of producing a desired effect. Such effects include, but are not limited to, enhanced survival, reduction in presence or severity of symptoms, reduced time to recovery, and prevention of initial disease.

As used herein, the terms “sample” and “biological sample” refer to any sample suitable for the methods provided by the present invention. In one embodiment, the biological sample of the present invention is a tissue sample, e.g., a biopsy specimen such as samples from needle biopsy (i.e., biopsy sample). In other embodiments, the biological sample of the present invention is a sample of bodily fluid, e.g., serum, plasma, sputum, lung aspirate, urine, and ejaculate.

Molecular Marker and Molecular Target polypeptides or polypeptide fragments also comprise amino acid sequences that are at least about 70% identical, preferably at least about 80% identical, more preferably at least about 90% identical and most preferably at least about 95% identical (e.g., 95%, 96%, 97%, 98%, 99%, 100%) to the mouse or human Molecular Marker and Molecular Target amino acid sequences shown in SEQ ID NO. 1 through SEQ ID NO. 21 with reference to sequences described above, are contemplated with respect to inhibiting Molecular Maker or Molecular Target expression and or function, when the comparison is performed by a BLAST algorithm wherein the parameters of the algorithm are selected to give the largest match between the respective sequences over the entire length of the respective reference sequences. Polypeptides comprising amino acid sequences that are at least about 70% similar, preferably at least about 80% similar, more preferably at least about 90% similar and most preferably at least about 95% similar (e.g., 95%, 96%, 97%, 98%, 99%, 100%) to any of the reference Molecular Marker and Molecular Target amino acid sequences when the comparison is performed with a BLAST algorithm wherein the parameters of the algorithm are selected to give the largest match between the respective sequences over the entire length of the respective reference sequences, are also included in constructs and methods of the present invention.

Sequence identity refers to the degree to which the amino acids of two polypeptides are the same at equivalent positions when the two sequences are optimally aligned. Sequence similarity includes identical residues and nonidentical, biochemically related amino acids. Biochemically related amino acids that share similar properties and may be interchangeable are discussed above.

“Homology” refers to sequence similarity between two polynucleotide sequences or between two polypeptide sequences when they are optimally aligned. When a position in both of the two compared sequences is occupied by the same base or amino acid monomer subunit, e.g., if a position in each of two DNA molecules is occupied by adenine, then the molecules are homologous at that position. The percent of homology is the number of homologous positions shared by the two sequences divided by the total number of positions compared times 100. For example, if 6 of 10 of the positions in two sequences are matched or homologous when the sequences are optimally aligned then the two sequences are 60% homologous. Generally, the comparison is made when two sequences are aligned to give maximum percent homology.

The phrase “selecting at least one of a group consisting of X and Y” refers to situations where X is selected alone, Y is selected alone, and where both X and Y are selected together.

### Novel Liver Disease Markers and Targets

The present invention provides novel molecular markers and molecular targets effective and useful to diagnose and monitor liver disease and useful to treat and discover new treatments for liver disease, including NAFLD, NASH and liver fibrosis. These novel marker and targets were discovered using differential gene expression of samples taken from individuals with and without NAFLD. Preferably, the differential gene expression will be determined on a per-cell-type basis, with the liver cell types being assayed comprising endothelial cells (ECs), hepatic stellate cells (hSCs), and Kupffer cells. In one embodiment of the present invention, liver tissue was collected from eight human donors. Three of the donors were rated as NAFLD low, with a NAFLD Activity Score of less than two while five of the donors were rated as NAFLD high, with a NAFLD Activity Score of greater than two. Liver tissue samples were isolated from each of the donors and separated into distinct cell types using methods standard in the art.

Sequence reads in Fast Q format were developed for four different cells types, as well as whole tissue (hereinafter referred to as the “five cell types”), of the eight donors, and were detected by IIlumina paired-end RNA-Seq sequencing of three technical replicates per sample. Initial raw quality assessment using the tool FastQC indicated sequence reads containing the IIlumina Universal Adapter Sequencing Primer, which had been subsequently clipped using the tool fast p. Technical replicates had been merged to obtain 80 Fast Q files (five cell types by eight donors by two read types: forward and reverse). The RNA-Seq aligner STAR (Version 2.6.0a) was used to map the reads to the human genome. Alignment was performed using the Homo sapiens reference genome sequence (Ensembl GRCh.38 primary assembly), using a transcript database (Ensembl GRCh.38.94) as a guide. The resulting aligned read (BAM) files had been checked for overall alignment quality. One file was excluded for technical reasons from all further analysis.

The BAM files were quantified on gene-level using the quantification tool Salmon (Version 0.12.0) to obtain a gene-level expression TPM table (transcripts per million), which was passed to the statistical analysis.

It will be apparent to those having skill in the art that the raw data produced by such a method is unlikely to be useful due to the large amount of variability present among the samples. As such, advanced statistical analysis of the samples is necessary to identify the key molecular targets that are differentially expresses between healthy and diseased patient samples. One embodiment of such a statistical analysis is present herewith as further described in Amezquita, R. A., Lun, A. T. L., Becht, E. et al. Orchestrating single-cell analysis with Bioconductor. Nat Methods (2019) doi:10.1038/s41592-019-0654-x.

In one embodiment, we selected all Kupffer samples from the merged counts table (counts) and the metadata table (metadata). We then used the differential expression tool EdgeR in R version 3.5.0 to calculate genes that were differentially expressed between samples with NAFLD low and NAFLD high donor samples. A DGEList was generated from counts (cds), then lowly detected genes were filtered out using the function (rowSums(1e+06*cds$counts/expandAsMatrix (cds$samples $lib.size, dim(cds))>1)>=3). We then calculated the normalization factors for each sample with calcNormFactors, followed by an estimation of common, tagwise, and trended dispersion with the functions estimateCommonDisp, estimateTagwiseDisp, and estimateTrendedDisp. Once all normalization procedures were complete we determined differential expression with the exactTest function. Multiple-testing was corrected for using the base R stats function p.adjust with the method set to “fdr”.

In another embodiment, we examined the clustering of all endothelial samples and identified that the samples from one individual were a significant outlier. This sample was excluded from the downstream analysis. We then selected all remaining endothelial samples from the merged counts table (counts) and the metadata table (metadata). We then used the differential expression tool EdgeR in R version 3.5.0 to calculate genes that were differentially expressed between NAFLD low and NAFLD high samples. A DGEList was generated from counts (cds), then lowly detected genes were filtered out using the function (rowSums(1e+06*cds$counts/expand AsMatrix(cds$samples$lib.size, dim(cds))>1)>=3). We then calculated the normalization factors for each sample with calcNorm Factors, followed by an estimation of common, tagwise, and trended dispersion with the functions estimateCommonDisp, estimateTagwiseDisp, and estimateTrendedDisp. Once all normalization procedures were complete we determined differential expression with the exactTest function. Multiple-testing was corrected for using the base R stats function p.adjust with the method set to “fdr”.

In another embodiment of the present invention, three tests were performed with the overlap between these tests being used for the final analysis. The three tests were: i) expression as a function of NAFLD high to NAFLD low (exact test) excluding Fibromyalgia patients; ii) expression as a function of NAFLD high (exact test) including all patients; and iii) expression as a function of weight (GLM test) including all patients.

We first selected all Stellate samples from the merged counts table (counts) and the metadata table (metadata). We then used the tool EdgeR in R version 3.5.0 to examine differential expression. A DGEList was generated from counts (cds), then lowly detected genes were filtered out using the function (rowSums(1e+06*cds$counts/expandAsMatrix(cds$samples $lib.size, dim(cds))>1)>=3). The normalization factors for each sample were calculated with calcNormFactors.

We next normalized gene expression based on dispersion estimates. For the first two exact tests, common, tagwise, and trended dispersions were estimated with the functions estimateCommonDisp, estimateTagwiseDisp, and estimateTrendedDisp. For the GLM test we estimated common and trended dispersion with estimateGLM CommonDisp and estimateGLMTrendedDisp. Once all normalization procedures were complete we determined differential expression for the first two exact tests with the exactTest function. Differential expression was determined for the GLM test by first fitting the model with glmQLFit and then testing the significance of the variable associated with weight. For all tests, multiple-testing was corrected for using the base R stats function p.adjust with the method set to “fdr”.

We then identified the gene overlap between all three tests and kept all genes that were significantly different between NAFLD-high and NAFLD-low independent of the inclusion of outliers into the model. We excluded all genes that were significant when the weight of the patient was included into the model.

### Novel Molecular Markers/Targets

Using the above-described methods, novel molecular markers/targets according to the present invention have been identified as being differentially expressed at a higher level in liver samples from patients with liver disease, more particularly NAFLD patients relative to samples from healthy individuals. A listing of these novel markers and targets according to the present invention is provided below.

TABLE 1
Differentially regulated genes in NAFLD-high samples.

| TARGET | Log₂ Fold Change | Fold Change In | |
|---|---|---|---|
| Name | In Expression | Expression | Liver Cell Type |
| LOXL4 | 1.54 | 2.91 | Endothelial |
| CHRM2 | 1.80 | 3.48 | Endothelial |
| DMKN | 2.04 | 4.11 | Endothelial |
| QPRT | 1.06 | 2.08 | Endothelial |
| SLC12A8 | 2.00 | 4.00 | Endothelial |

### Molecular Marker/Target LOXL4:

In certain embodiments of the present invention, the novel target for modulation of liver disease is encoded by the gene LOXL4. Other aliases for the LOXL4 gene include Lysyl Oxidase Like 4, Lysyl Oxidase-Related Protein C, Lysyl Oxidase-Like Protein 4, LOXC, Lysyl Oxidase-Like 4 Pseudogene, Lysyl Oxidase Homolog 4, Lysyl Oxidase Related C, and Lysyl Oxidase-Like 4.

The LOXL4 gene encodes one or more proteins that are members of the lysyl oxidase gene family. Lysyl oxidases are a family of at least five copper-dependent amine oxidases including LOX, LOXL, LOXL2 , LOXL3 and LOXL4 that catalyze the oxidation of peptidyl lysine to δ-aminoadipic β-semialdehyde, the intermediate precursor during the formation of covalent cross-linkages that stabilize fibers of elastin and collagen, and contributes to the development and maintenance of the extracellular matrix (Maki, J. M., et al., Cloning and characterization of a fifth human lysyl oxidase isoenzyme: the third member of the lysyl oxidase-related subfamily with four scavenger receptor cysteine-rich domains. Matrix Biol. 20: 493-496, 2001). A highly conserved amino acid sequence at the C-terminus end appears to be sufficient for amine oxidase activity, suggesting that each family member may retain this function. The N-terminus is poorly conserved and may impart additional roles in developmental regulation, senescence, tumor suppression, cell growth control, and chemotaxis to each member of the family.

LOXL4 expression has been associated with several human tumor cells, including melanoma cells, fibrosarcoma and rhabdosarcoma cells, and in prostate- and breast carcinoma. It has been found in both extracellular and intracellular locations, and has multiple functions in stromal and epithelial cells and tissues e. g., maturation of fibrillar matrix proteins in fibrosing processes and dictates their stability against metalloproteinases. LOXL4 and its gene product are most strongly associated with squamous cell carcinoma of the head and neck, where upregulation of the gene as well as overexpression of the gene product have been observed. The aberrant expression of LOXL4 in such cancerous cells has made it a focus of research as both a marker of such cancers and as a target for treating the same (see US Pat. Pub. No. 20170253668A1).

In certain embodiments, LOXL4 encodes a 756 amino acid LOXL4 protein as provided in SEQ ID NO. 1. LOXL4 also includes functionally equivalent protein sequences having amino acid sequences at least about 70% similar, preferably at least about 80% similar, more preferably at least about 90% similar and most preferably at least about 95% similar (e.g., 95%, 96%, 97%, 98%, 99%, 100%) homologous to SEQ ID NO. 1.

Two alternative splice variants of LOXL4 have been identified. The spliced segments were exon 9 (splice variant 1) or both exons 8 and 9 (splice variant 2). In ovarian carcinoma, splice variant 1 was significantly elevated in effusions compared to solid lesions (p<0.001). Splice variant 2 appeared only in effusions. In breast carcinoma, LOXL4 was expressed only in the effusion samples. (Sebban S, Davidson B, Reich R. Lysyl oxidase-like 4 is alternatively spliced in an anatomic site-specific manner in tumors involving the serosal cavities. Virchows Arch. 2009 January; 454(1):71-9.)

As noted above, LOXL4 overexpression has been associated with various human cancers. Furthermore, the LOXL4 protein has been detected in various human tissues, including cerebral cortex, bone marrow, spleen, lung, and kidney (see the summary of known human tissue expression from the Human Protein Atlas available at https://www.proteinatlas.org/ENSG00000138131-LOXL4/tissue). To date, elevated levels of LOXL4 expression have not been detected in any of the cell types of the liver, and no data currently links LOXL4 overexpression with diseases of the liver.

Thus, it was surprising to discover that LOXL4 expression demonstrates at least a 2.91 (Log2(1.544020069)) fold change increase in the endothelial cells of diseased liver tissue as compared to healthy tissue (see Table 1), as further discussed herein. As such, in certain embodiments of the present invention, LOXL4 is a new molecular marker and molecular target in the diagnosis and treatment of diseases of the liver, including, but not limited to, NAFLD, NASH, fibrosis of the liver and related conditions.

There are several known pharmaceutical modulators of LOXL4 including, by way of example and not limitation, the following compounds:

The pan-lox inhibitor Beta-am inopropionitrile (BAPN). BAPN is the toxic constituent of peas from Lathyrus plants, e.g., lathyrus odoratus. Lathyrism, a disease known for centuries, encompasses two distinct entities: a disorder of the nervous system (neurolathyrism) leading to limb paralysis, and a disorder of connective tissue, causing either bone deformity (osteolathyrism) or aortic aneurisms (angiolathyrim). BAPN causes osteolathyrism and angiolathyrism when ingested in large quantities. BAPN is under investigation as a treatment for certain cancers. BAPN has the following chemical structure.

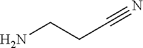

The pan-lox inhibitor Thiram. Thiram is most commonly used as fungicide in agricultural contexts. Ingestion in humans can cause headaches, dizziness, fatigue, nausea, and diarrhea, however Thiram has been used as a topical treatment as an anti-fungal agent and as a treatment to prevent post-operative surgical infections. Thiram has the following chemical formula:

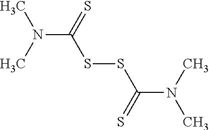

The pan-lox inhibitor Disulfiram. Disulfiram was initially utilized for industrial purposes in the late 19^{th} century, mainly in the Sulphur vulcanization of rubber. However, it was also discovered that Disulfiram possessed many medicinal properties, and as such it was investigated as a treatment for many diseases of the time, including scabies, parasitic skin infections, and intestinal worms. Today, Disulfiram is primarily used as a treatment for chronic alcoholism under the trade name Antabuse, as it inhibits the action of the acetaldehyde dehydrogenase enzyme within the patient. Doing so increases the severity of “hang-over” symptoms of the patient and causes them to be experienced almost immediately after alcohol is consumed, thus reducing the patient's desire to drink alcoholic beverages. Disulfiram has the following chemical structure:

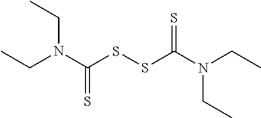 [SMILES: CCN(CC)C(=S)SSC(=S)N(CC)CC]

Additional modulators of LOXL4 include those discussed in Hajdú I et al., Bioorg Med Chem Lett. 2018 Oct. 1; 28(18):3113-3118. doi: 10.1016/j.bmc1.2018.07.001. Epub 2018 Jul. 6, Inhibition of the lox enzyme family members with old and new ligands. selectivity analysis revisited, Bioorganic & Medicinal Chemistry Letters (2018), doi: https://doi.org/10.1016/j.bmc1.2018.07.001 and others known in the art or later developed.

Thus, in certain embodiments of the present invention, LOXL4 is modulated in order to provide therapeutic efficacy to a patient suffering from liver disease, including, but not limited to NAFLD, NASH, liver fibrosis and related conditions. In certain other embodiments, a therapeutically effective amount of a LOXL4 modulator is administered to a patient to treat and/or prevent the progression of liver disease. Such a LOXL4 modulator may be selected from a list comprising BAPN, Thiram, Disulfiram, TGX-L255 and TGX-L124. In alternate embodiments, the modulation of LOXL4 may be accomplished by, for example, altering the expression level of the LOXL4 gene. In yet another embodiment, LOXL4 activity is modulated indirectly, for example, by altering the activity of cellular process that work in concert with, or in opposition to, the activity of LOXL4.

### Molecular Marker/Target CHRM2:

In certain embodiments of the present invention, the protein target for modulation is encoded by the gene CHRM2. This gene encodes the protein ACM. This gene also has additional aliases: Cholinergic Receptor Muscarinic 2, Acetylcholine Receptor, Muscarinic 2, Muscarinic Acetylcholine Receptor M2, Cholinergic Receptor, Muscarinic 2, Muscarinic M2 Receptor, 7TM Receptor, and HM2.

Alternate identification information for the CHRM2 gene are as follows: HGNC: 1951, Entrez Gene: 1129, Ensembl: ENSG00000181072, OMIM: 118493, UniProtKB: P08172.

ACM is a muscarinic cholinergic receptor. The muscarinic cholinergic receptors belong to a larger family of G protein-coupled receptors. The functional diversity of these receptors is defined by the binding of acetylcholine to these receptors and includes cellular responses such as adenylate cyclase inhibition, phosphoinositide degeneration, and potassium channel mediation. Muscarinic receptors influence many effects of acetylcholine in the central and peripheral nervous system. The muscarinic cholinergic receptor 2 is involved in mediation of bradycardia and a decrease in cardiac contractility. The muscarinic acetylcholine receptor mediates various cellular responses, including inhibition of adenylate cyclase, breakdown of phosphoinositides and modulation of potassium channels through the action of G proteins. Primary transducing effect is adenylate cyclase inhibition. Signaling promotes phospholipase C activity, leading to the release of inositol trisphosphate (IP3); this then triggers calcium ion release into the cytosol.

Multiple alternatively spliced transcript variants have been described for this gene. [provided by RefSeq, July 2008].

Diseases associated with CHRM2 include Intestinal Schistosomiasis and Major Depressive Disorder.

Muscarinic receptors are widely distributed throughout the body and control distinct functions according to location and subtype (M1-M5). They are predominantly expressed in the parasympathetic nervous system where they exert both inhibitory and excitatory effects.

Cholinergic receptor muscarinic 2 (muscarinic acetylcholine receptor M2) acetylcholine receptor (Gi/Go-coupled) with known agonist and antagonists. Acetylcholine induces fibrogenic effects via M2/M3 acetylcholine receptors in non-alcoholic steatohepatitis and in primary human hepatic stellate cells. However, there has been no suggestions that CHRM2 expression plays a role in NAFLD or other liver disease progression in liver endothelial cells (ECs).

The muscarinic acetylcholine receptor mediates various cellular responses, including inhibition of adenylate cyclase, breakdown of phosphoinositides and modulation of potassium channels through the action of G proteins. Primary transducing effect is adenylate cyclase inhibition. Signaling promotes phospholipase C activity, leading to the release of inositol trisphosphate (IP3); this then triggers calcium ion release into the cytosol.

Five mammalian CHRM subtypes (CHRM1-5) are described; odd-numbered CHRM (CHRM1, 3, and 5) preferentially activate G_{q/11} proteins, whereas even-numbered CHRM (CHRM2 and 4) activate G_{l/o} proteins.

In certain embodiments, the CHRM2 gene encodes a protein with the 466 amino acid sequence shown at SEQ ID NO. 2. CHRM2 also includes functionally equivalent protein sequences having amino acid sequences at least about 70% similar, preferably at least about 80% similar, more preferably at least about 90% similar and most preferably at least about 95% similar (e.g., 95%, 96%, 97%, 98%, 99%, 100%) homologous to SEQ ID NO. 2.

According to prior work, when primary human hSCs were analyzed for synthesis of endogenous ACh and acetylcholin-esterase and gene expression of choline acetyltransferase and muscarinic ACh receptors it was found that human hepatic stellate cells synthesize ACh and acetylcholinesterase and express choline acetyltransferase and M1-M5 mAChR. The M2 receptor was increased during NASH progression measuring whole liver biopsies, while both M2 and M3 were found up-regulated in activated hHSC. (Morgan M L et al., J Gastroenterol Hepatol. 2016 February; 31(2):475-83. doi: 10.1111/jgh.13085. PubMed PMID: 26270240). Interestingly, work in rats showed that the M2 receptor decreased during CCL4 induced fibrosis (Luo L et al., Exp Toxicol Pathol. 2017 February; 69(2):73-81. doi: 10.1016/j.etp.2016.11.005. Epub 2016 Nov. 26. PubMed PMID: 27899232).

In contrast with these prior results, the methods of the present invention have made the surprising discovery that was no apparent regulation in human stellate cells nor in human whole tissue. Instead the up-regulation due to disease was occurring in liver endothelial cells with a fold change of 3.48 (Log2(1.8)) times over baseline. As such, in certain embodiments of the present invention, CHRM2 is a new molecular marker and molecular target in the diagnosis and treatment of diseases of the liver, including, but not limited to, NAFLD, NASH, fibrosis of the liver and related conditions.

A number of pharmaceutical compounds have been developed to inhibit CHRM2 as a possible treatment for various neurological disorders. Such compounds include oxybutynin, oxybutynin chloride, oxybutynin+pilocarpine, trospium chloride+xanomeline, and the tetrahydrofuranic compound AVANEX-141.

Thus, in certain embodiments of the present invention, CHRM2 is modulated in order to provide therapeutic efficacy to a patient. In certain embodiments, a therapeutically efficacious amount of a CHRM2 modulator is administered to a patient to treat liver disease, selected from the group consisting of NAFLD, NASH, liver fibrosis and related conditions. Such a CHRM2 modulator may be selected from a list comprising oxybutynin, oxybutynin chloride, oxybutynin+pilocarpine, trospium chloride+xanomeline, AVANEX-141, or other known or as-yet undiscovered modulators of CHRM2. In alternate embodiments, the modulation of CHRM2 may be accomplished by, for example, altering the expression level of the CHRM2 gene. In yet another embodiment, CHRM2 activity is modulated indirectly, for example, by altering the activity of cellular process that work in concert with, or in opposition to, the activity of CHRM2.

### Molecular Marker/Target DMKN:

In certain embodiments of the present invention, the protein target for modulation is encoded by the gene DMKN. Other aliases for the DMKN gene include Epidermis-Specific Secreted Protein SK30/SK89, ZD52F10, UNQ729.

DMKN is up regulated in inflammatory diseases, and it was first observed as expressed in the differentiated layers of the skin. The most interesting aspect of this gene is the differential use of promoters and terminators to generate isoforms with unique cellular distributions and domain components. There are two major DMKN protein isoforms: a 90 amino acid isoform 1 (SEQ ID NO. 3) and a 3 76 amino acid isoform 2 (SEQ ID NO. 4). DMKN comprising isoform 1 and isoform 2 also includes functionally equivalent protein sequences having amino acid sequences at least about 70% similar, preferably at least about 80% similar, more preferably at least about 90% similar and most preferably at least about 95% similar (e.g., 95%, 96%, 97%, 98%, 99%, 100%) homologous to SEQ ID NO. 3 and SEQ ID NO. 4.

Alternatively, spliced transcript variants encoding different isoforms have been identified for this gene. DMKN is alternatively spliced into at least 14 additional mRNA transcripts that encode 13 additional protein isoforms (See SEQ ID NO. 5 to SEQ ID NO. 17) and also includes functionally equivalent protein sequences having amino acid sequences at least about 70% similar, preferably at least about 80% similar, more preferably at least about 90% similar and most preferably at least about 95% similar (e.g., 95%, 96%, 97%, 98%, 99%, 100%) homologous to SEQ ID NO. 5 to SEQ ID NO. 17., described below. Major isoforms expressed in keratinocytes are involved in terminal differentiation of epidermis (Toulza, E., et.al., J. Invest. Derm. 126: 503-506, 2006), whereas a ubiquitously expressed intracellular isoform is involved in early endosome trafficking (Leclerc, E. A., et.al., PLoS One 6: e17816, 2011).

Matsui, T. et.al., Genomics 84: 384-397, 2004 cloned 2 splice variants of DMKN, which they called alpha and beta, from human and mouse skin cDNA libraries. The deduced mouse and human DMKN-alpha proteins contain 104 and 88 amino acids, respectively. The deduced mouse and human DMKN-beta proteins contain 517 and 476 amino acids, respectively. All mouse and human proteins have an N-terminal signal sequence, a myristoylation site, and a C-terminal domain common to alpha and beta isoforms. In addition, the beta isoforms have a central glycine- and serine-rich domain, 2 cysteines, and a potential N-glycosylation site. Northern blot analysis of mouse tissues detected DMKN transcripts of 0.6 (alpha) and 2.0 (beta) kb in skin, with weaker expression in stomach, and very low expression in lung. Quantitative RT-PCR detected DMKN in stratified epithelia from several mouse tissues and also in trachea, urinary bladder, and thymus. Notably, DMKN was undetectable in typical simple epithelia. In situ hybridization of mouse foot pad detected DMKN-alpha and DMKN-beta in spinous layer. In mouse, DMKN expression began late in embryonic development, concomitant with stratification of the epidermis. Human DMKN-alpha and -beta were N-terminally processed and secreted following expression in 293/EBNA-1 cells. DMKN expression was induced at terminal differentiation in cultured human keratinocytes.

From human epidermis cDNA library, Toulza et al. (2006) obtained 13 dermokine clones that revealed 3 transcription start sites, 2 transcription termination sites, and 8 alternative coding exons. In addition to alpha and beta forms, Toulza et al. (2006) identified 2 gamma variants and 9 delta variants. The transcripts potentially encoded 10 different proteins, and all except those translated from delta splice variants, were predicted to have an N-terminal signal sequence and be secreted. Delta isoforms were predicted to be cytosolic. PCR analysis of 17 human tissues detected beta and gamma variants in epidermis only, alpha variant in epidermis and placenta, and delta variants in most tissues examined. Epitope-tagged dermokines alpha, beta, and gamma-2 were detected in the culture medium of transfected cells whereas delta-5 dermokine remained intracellular. Immunohistochemical analysis detected beta/gamma dermokine in the granular layer, predominantly at the apical edge of keratinocytes. In lesional skin of psoriatic patients, these isoforms were more widely expressed than in normal skin. Immunoelectron microscopy localized beta/gamma dermokine in small cytoplasmic keratinosomes and in the edge of extracellular space between uppermost granular cell and lowermost corneocytes. Western blot analysis of normal human epidermis detected 2 main bands at apparent molecular masses of 66 (beta) and 45 (gamma) kD.

Naso, M. F. et.al., J. Invest. Derm. 127: 1622-1631, 2007 showed that full-length human DMKN-beta contains an N-terminal signal peptide, followed by a globular domain (encoded by exon 1), a coiled-coil collagen-like domain (exon 2), a serine- and glycine-rich keratin-like domain (exons 4-8), a spacer region (exons 9-17), and a C-terminal globular domain (exons 19-23). DMKN-alpha is essentially the C-terminal globular domain of the beta isoform, whereas gamma isoforms lack the C-terminal globular domain, delta isoforms lack N-terminal domains, and the epsilon isoform includes only part of the keratin-like domain and spacer region. In situ hybridization and immunohistochemical analysis of human tissues revealed expression of several DMKN isoforms in tissues other than skin.

Leclerc, E. A., et.al., J. Cell Sci. 127: 2862-2872, 2014 found that splicing of mouse DMKN is less complex than that of human DMKN. RT-PCR of newborn mouse epidermis identified 8 splice variants within the alpha, beta, and gamma DMKN families. No delta splice variant was identified.

Using yeast 2-hybrid screens, Leclerc et al. (2011) found that the 137-amino acid DMKN delta-5 protein interacted with the small GTPases RABSA (179512), RABSB (179514), and RABSC (604037). Protein pull-down experiments confirmed the interaction between DMKN delta-5 and endogenous HeLa cell RABS. Transient expression of DMKN delta-5 in HeLa cells led to formation of punctate structures that colocalized with endogenous RABS and clathrin (see 118960), indicating that DMKN delta-5 is involved in early steps of endocytosis. DMKN delta-5 also colocalized with transferrin (190000) at early stages of endocytosis, but did not alter its endocytosis or recycling kinetics. DMKN delta-5 interacted with both inactive GDP-bound and active GTP-bound forms of RABS in vitro, but preferentially targeted GDP-bound forms in HeLa cells. DMKN delta-5 appeared to enhance GDP-GTP exchange on RABS, leading to enlargement of RABS-positive vesicles in cotransfected cells. Leclerc et al. (2011) concluded that DMKN delta-5 is involved in early endosomal vesicle formation and trafficking by promoting GTP loading onto RAB5.

Higashi, K., et.al., FEBS Lett. 586: 2300-2305, 2012 observed that the C-terminal domain of dermokine-beta shares a high degree of similarity with cytokines. They found that recombinant full-length human dermokine-beta or its isolated C-terminal domain inhibited phosphorylation of ERK1 (MAPK3; 601795)/ERK2 (MAPK1; 176948) and elevated caspase (see CASP3, 600636) activity in normal human keratinocytes. Pharmacologic inhibition of ERK signaling increased expression of dermokine-beta/gamma and dermokine-alpha. Chemical crosslinking and immunoprecipitation studies showed that the C-terminal domain of dermokine-beta directly interacted with GRP78 (HSPAS; 138120), a receptor for cell-surface ligands. Knockdown of GRP78 via short interfering RNAs abrogated dermokine-beta effects on inhibition ERK phosphorylation, caspase activation, and dermokine gene expression. Higashi et al. (2012) concluded that dermokine-beta acts through GRP78 to inhibit ERK signaling in keratinocytes.

DMKN protein has been detected in a limited number of human tissues, with greatest expression observed in skin (see the summary of known human tissue expression from the Human Protein Atlas available at https://www.proteinatlas.org/ENSG00000161249-DMKN/tissue). To date, DMKN protein expression has only been detected in hepatocytes of normal human liver (Naso et al. 2007). DMKN upregulation was observed in mouse liver following exposure to CC14 (Lefebvre et al. 2017) however various transcriptome studies investigating gene expression alterations in the livers of patients with NAFLD, NASH or cirrhosis did not detect DMKN in human liver (Lefebvre P, et.al., JCI Insight. 2017 Jul. 6; 2(13); Chan K M, et.al.,. Kaohsiung J Med Sci. 2016 April; 32(4):165-76; Gerhard G S, et.al., J Endocr Soc. 2018 Jun. 5; 2(7):710-726; Ryaboshapkina M, and Hammar M., Sci Rep. 2017 Sep. 27; 7(1):12361).

Thus, it was surprising to discover that DMKN gene expression demonstrates a 4.11 (Log2 (2.037) times fold change increase in the endothelial cells of diseased liver tissue as compared to healthy tissue (see Table 1, as discussed in the methods section above). As such, in certain embodiments of the present invention, DMKN is targeted as a method of treating liver disease. As such, in certain embodiments of the present invention, DMKN is a new molecular marker and molecular target in the diagnosis and treatment of diseases of the liver, including, but not limited to, NAFLD, NASH, fibrosis of the liver and related conditions.

Thus, in certain embodiments of the present invention, DMKN is modulated in order to provide therapeutic efficacy to a patient. In certain embodiments, a therapeutically efficacious amount of a DMKN modulator is administered to a patient to treat liver diseases, including, but not limited to, NAFLD, NASH, liver fibrosis and related conditions. Such a DMKN modulator may be selected from a list comprising any known or as-yet undiscovered modulators of DMKN. In alternate embodiments, the modulation of DMKN may be accomplished by, for example, altering the expression level of the DMKN gene. In yet another embodiment, DMKN activity is modulated indirectly, for example, by altering the activity of cellular process that works in concert with, or in opposition to, the activity of DMKN.

### Molecular Marker/Target QPRT:

In certain embodiments of the present invention, the target for modulation is encoded by the gene QPRT. Other aliases for the QPRT include Quinolinate Phosphoribosyltransferase, Nicotinate-Nucleotide Pyrophosphorylase (Carboxylating), QPRTase, Nicotinate-Nucleotide Pyrophosphorylase [Carboxylating], Quinolinate Phosphoribosyltransferase [Decarboxylating], Epididym is Secretory Sperm Binding Protein Li 90n, EC 2.4.2.19, HEL-S-90n, and QAPRTase.

The QPRT mRNA and protein levels are high in kidney and liver, and QPRT is expressed at lower levels in a number of other tissues (see databases for the NCBI, https://www.ncbi.nlm.nih.gov/gene/23475#gene-expression, and for Protein Atlas, https://www.proteinatlas.org/ENSG00000103485-QPRT/tissue). Expression of QPRT is increased in monocytes stimulated with interferon-y, suggesting implications for neurodegenerative and inflammatory disease (Jones S P, et.al., PLoS One. 2015 Jun. 26; 10(6):e0131389. doi: 10.1371/journal.pone.0131389). QPRT expression is also implicated in poor prognosis for glioma (Sahm F, et.al.,Cancer Res. 2013 Jun 1; 73(11):3225-34). Importantly with respect to liver disease, one group has reported that QPRT is expressed at lower levels in NAFLD patients (Hotta K, et.al., Hepatol Res. 2018 February; 48(3):E320-E334).

In certain embodiments, QPRT encodes a protein with the following sequence SEQ ID NO 18 and isoforms shown by SEQ ID NO. 19 and SEQ ID NO. 20. QPRT and these isoforms (SEQ ID NO. 18 through SEQ ID NO. 20) also includes functionally equivalent protein sequences having amino acid sequences at least about 70% similar, preferably at least about 80% similar, more preferably at least about 90% similar and most preferably at least about 95% similar (e.g., 95%, 96%, 97%, 98%, 99%, 100%) homologous to SEQ ID NO. 18 through SEQ ID NO. 20.

The QPRT gene encodes a key enzyme in catabolism of quinolinate, an intermediate in the tryptophan-nicotinamide adenine dinucleotide pathway. This pathway is used for the production of NAD+, a cofactor for many metabolic enzymes. Lowered levels of NAD+ are found in many metabolic disorders including NAFLD (Elhassan Y S, et.al., J Endocr Soc. 2017 May 15; 1(7):816-835).

Surprisingly, the methods of the present invention have identified QPRT as being expressed significantly higher by 2.8 (Log2(1.06259821) times fold in liver endothelial cells in NAFLD patients, suggesting that NAD+ levels may be lower in these cells. Our findings also show that there are no differences in QPRT levels in whole liver tissue and hepatocytes in normal versus NAFLD patients, in contrast to a report that QPRT is expressed at lower levels in NAFLD patients (Hotta et al, 2018). Therefore, our findings are novel and as such, in certain embodiments of the present invention, QPRT is targeted as a method of treating liver disease. As such, in certain embodiments of the present invention, QPRT is a new molecular marker and molecular target in the diagnosis and treatment of diseases of the liver, including, but not limited to, NAFLD, NASH and fibrosis of the liver.

Known pharmaceutical modulators of QPRT include, by way of example and not limitation, the following compounds:

Phthalic acid (1,2-Benzenedicarboxylic acid), an inhibitor of QPRT. Phthalic acid treatment of primary neurons and astrocytes reduced cell viability (Braidy et al, 2011), indicating that QPRT activity and intracellular NAD+is important in the function of the nervous system.

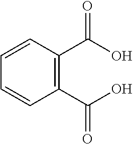 [SMILES: O=C(O)c1ccccc1C(=O)O]

Clofibrate, a PPARa agonist and lipid lowering agent, stimulates QPRT expression and increases NAD+ levels in rat hepatocytes (Shin M, et.al., Adv Exp Med Biol. 1999; 467:333-40), but failed to improve the symptoms of NASH patients in a clinical trial (Laurin J, et al., Hepatology. 1996 June; 23(6):1464-7).

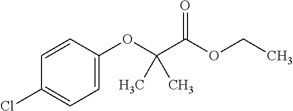 [SMILES: CCOC(=O)C(C)(C)Oc1ccc(Cl)cc1]

Thus, in certain embodiments of the present invention, QPRT is modulated in order to provide therapeutic efficacy to a patient. In certain embodiments, a therapeutically efficacious amount of a QPRT modulator is administered to a patient to treat liver diseases, including, but not limited to, NAFLD, NASH, liver fibrosis and related conditions. Such a QPRT modulator may be selected from a list comprising Phthalic acid, Clofibrate, or other known or as-yet undiscovered modulators of QPRT. In alternate embodiments, the modulation of QPRT may be accomplished by, for example, altering the expression level of the QPRT gene. In yet another embodiment, QPRT activity is modulated indirectly, for example, by altering the activity of cellular process that work in concert with, or in opposition to, the activity of QPRT.

### Molecular Marker/Target SLC12A8:

In certain embodiments of the present invention, the target for modulation is encoded by the gene SLC12A8. Other aliases for the SLC12A8 gene include polypeptide solute carrier family 12 member 8; Cation-Chloride Cotransporter 9; Solute Carrier Family 12 (Sodium/Potassium/Chloride Transporters), Member 8; and CCC9.

The SLC12A8 gene encodes a member of the cation-chloride cotransporter (CCC) family. The best characterized members of this family transport sodium, potassium, and chloride ions in the kidneys and are the targets of the thiazide and loop diuretics (SLC12A1/NKCC2; Gagnon K B, Delpire E., Am J Physiol Cell Physiol. 2013 Apr. 15; 304(8):C693-714). However, SLC12A8/CCC9 has been shown to transport the polyamines sperm idine, spermine, and putrescine which is enhanced in the presence of certain amino acids and unaffected by inorganic ions (Daigle N D, et.al., J Cell Physiol. 2009 September; 220(3):680-9). The gene is a candidate for psoriasis susceptibility (Hüffmeier U, et.al., J Invest Dermatol. 2005 November; 125(5):906-12) and has been found to be genetically associated with breast cancer survival (Kim J E, et.al., Pharmacogenomics J. 2018 September; 18(5):633-645.). Expression of mRNA (https://www.ncbi.nlm.nih.gov/gene/84561) and protein (https://www.proteinatlas.orq/ ENSG00000221955-SLC12A8/tissue) show that SLC12A8 is expressed in most tissues.

In certain embodiments, SLC12A8 mRNA transcript encodes a protein with SEQ ID NO. 21. SLC12A8 also includes functionally equivalent protein sequences having amino acid sequences at least about 70% similar, preferably at least about 80% similar, more preferably at least about 90% similar and most preferably at least about 95% similar (e.g., 95%, 96%, 97%, 98%, 99%, 100%) homologous to SEQ ID NO. 21.

Surprisingly in light of the above-described art, we have discovered that SLC12A8 expression is significantly elevated by 4 (Log2(1.99504613)) times fold in the liver endothelial cells (LEC) of NAFLD-diseased liver tissue compared to healthy liver tissue (see Table 1), as discussed in the methods section above. Since there are no publications or patents on the role and expression of SLC12A8 in LEC or in liver disease, it was surprising to discover that SLC12A8 is expressed in LEC. As such, in certain embodiments of the present invention, SLC12A8 is targeted as a method of diagnosing and treating liver disease.

Known pharmaceutical modulators of SLC12A8 include, by way of example and not limitation, the following compounds:

Inhibitors of SLC12A8 include pentamidine, methyl-glyoxal-bisguanyl-hydrazone (MGBG), and furosemide (Daigle et al, 2009):

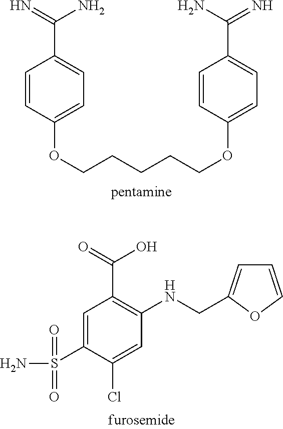 [SMILES: N=C(N)c1ccc(OCCCCCOc2ccc(C(=N)N)cc2)cc1 ; NS(=O)(=O)c1cc(C(=O)O)c(NCc2ccco2)cc1Cl]

Enhancers of SLC12A8 include glutamate and aspartate, but not other amino acids (Daigle et al, 2009).

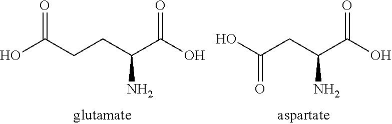

Thus, in certain embodiments of the present invention, SLC12A8 is modulated in order to provide therapeutic efficacy to a patient. In certain embodiments, a therapeutically efficacious amount of a SLC12A8 modulator is administered to a patient. Such a SLC12A8 modulator may be selected from a list comprising pentamidine, MGBG, furosemide, glutamate, aspartate, or other known or as-yet undiscovered modulators of SLC12A8. In alternate embodiments, the modulation of SLC12A8 may be accomplished by, for example, altering the expression level of the SLC12A8 gene. In yet another embodiment, SLC12A8 activity is modulated indirectly, for example, by altering the activity of cellular process that work in concert with, or in opposition to, the activity of SLC12A8.

### Screening for Modulators of Molecular Targets:

The invention also provides a method of determining whether NASH, NAFLD, liver fibrosis or related conditions in a given subject is amenable to treatment with a modulator of a molecular target as disclosed herein. The method can be performed, for example, by measuring the expression or activity of the molecular target cell sample or serum sample of a subject to be treated, and determining that the molecular target activity or expression is elevated or abnormally elevated as compared to the level of molecular target activity or expression in corresponding normal cells or control serum, which can be a sample of normal cells of the subject. Detection of elevated or abnormally elevated level of the molecular target activity or expression in the cells as compared to the corresponding normal cells indicates that the subject can benefit from treatment with a modulator of the molecular target. A sample of cells used in the present method can be obtained using a biopsy procedure (e.g., a needle biopsy), or can be a sample of cells obtained by a medical procedure.

The method of identifying NASH, NAFLD, liver fibrosis or related conditions amenable to treatment with a modulator of a molecular target according to the present invention can further include contacting cells of the sample with at least one test modulator, and detecting a change in the molecular target activity or expression in the cells following said contact. Such a method provides a means to confirm that such liver diseases are amenable to treatment with a modulator of the molecular target. Further, the method can include testing one or more different test modulators, either alone or in combination, thus providing a means to identify one or more test modulators useful for treating the particular liver disease being examined. Accordingly, the present invention also provides a method of identifying a modulator useful for treating NASH, NAFLD, liver fibrosis or related conditions in a subject, especially a human patient.

In another aspect, the invention provides a method of detecting liver disease in a subject and/or confirming a diagnosis of such liver disease in the subject. The method includes detecting and/or diagnosing liver disease in a subject.

In another aspect, the present invention provides a method of identifying a modulator useful for treating liver disease in a patient. The method includes contacting a sample of cells with at least one test modulator, wherein a change in molecular target activity or expression in the presence of the test modulator as compared to molecular target activity or expression in the absence of the test modulator identifies the modulator as useful for treating NASH, NAFLD, liver fibrosis and related conditions. Thus, the invention likewise provides a method of screening for molecular target modulators.

When practiced as an in vitro assay, the methods can be adapted to a high throughput format, thus allowing the examination of a plurality (i.e., 2, 3, 4, or more) of cell samples and/or test modulators, which independently can be the same or different, in parallel. A high throughput format provides numerous advantages, including that test modulators can be tested on several samples of cells from a single patient, thus allowing, for example, for the identification of a particularly effective concentration of an modulator to be administered to the subject, or for the identification of a particularly effective modulator to be administered to the subject. Alternatively, or in addition thereto, the high throughput format may be used to screen for molecular target modulators using a report in cells transfected with molecular target(s) with or without expression vectors.

As such, a high throughput format allows for the examination of two, three, four, etc., different test agents, alone or in combination, on the hepatocellular carcinoma or NASH cells of a subject such that the best (most effective) agent or combination of agents can be used for a therapeutic procedure. Further, a high throughput format allows, for example, control samples (positive controls and or negative controls) to be run in parallel with test samples, including, for example, samples of cells known to be effectively treated with an agent being tested.

A high throughput method of the invention can be practiced in any of a variety of ways. For example, different samples of cells obtained from different subjects can be examined, in parallel, with same or different amounts of one or a plurality of test modulator(s); or two or more samples of cells obtained from one subject can be examined with same or different amounts of one or a plurality of test modulators. In addition, cell samples, which can be of the same or different subjects, can be examined using combinations of test modulators and/or known effective agents. Variations of these exemplified formats also can be used to identify a modulator or combination of modulators useful for treating liver diseases having elevated molecular target activity or expression.

When performed in a high throughput (or ultra-high throughput) format, the method can be performed on a solid support (e.g. , a microtiter plate, a silicon wafer, or a glass slide), wherein samples to be contacted with a test modulator are positioned such that each is delineated from each other (e.g. , in wells). Any number of samples (e.g., 96, 1024, 10,000, 100,000, or more) can be examined in parallel using such a method, depending on the particular support used. Where samples are positioned in an array (i.e., a defined pattern), each sample in the array can be defined by its position (e.g., using an x-y axis), thus providing an “address” for each sample. An advantage of using an addressable array format is that the method can be automated, in whole or in part, such that cell samples, reagents, test agents, and the like, can be dispensed to (or removed from) specified positions at desired times, and samples (or aliquots) can be monitored, for example, for molecular target activity or expression and/or cell viability.

### Molecular Markers Diagnostic Uses:

The expression of elevated levels of molecular marker nucleic acids may be detected for diagnosis or detection of liver diseases, or predisposition to such a condition. Such a molecular marker test would utilize a liver biopsy in order to obtain a suitable patient test sample. As provided herein the data indicates that in humans with liver disease, including NAFLD, NASH, liver fibrosis and related conditions, molecular modulator protein levels are elevated in the range of 2-5 fold above normal. A bank of human healthy liver specimens would provide an average baseline immunoblot signal using densitometry quantification based on β-actin load. Values>1.5-fold greater than the averaged baseline would be indicative of a diseased liver condition, or likelihood of progression to NAFLD, NASH, liver fibrosis and related conditions. Methods including IHC would be useful in detecting elevated such molecular marker levels. The data described herein indicate that the molecular marker nucleic acid levels or protein liver levels would be a good marker of risk for progression of benign steatosis to clinically significant NAFLD and NASH. RNAseq is another method that may be useful for such testing. Additionally, monitoring samples for elevated levels of molecular marker nucleic acids in patients undergoing treatments as described herein, may provide an indication of treatment efficacy and/or effectiveness. Molecular marker nucleic acids may also be used for the expression or production of molecular marker polypeptides.

Accordingly, the present invention also relates to a method for diagnosing liver disease or susceptibility to liver disease in a human subject comprising: (a) performing an in vitro nucleic acid detection assay on a nucleic acid sample from a human subject to detect the presence of an elevated level of a molecular marker of the present invention in the subject's nucleic acid sample when compared to a control molecular marker level and (b) diagnosing the subject as being susceptible to or having liver disease (NAFLD, NASH and/or liver fibrosis) based on an elevated level of molecular marker in the subject's nucleic acid sample, wherein the nucleic acid detection assay comprises amplification of a nucleic acid molecule with at least a primer pair, said primer pair comprising a forward primer comprising a nucleotide sequence and a reverse primer nucleotide sequence that hybridizes to molecular maker to produce amplified molecular marker nucleic acid, wherein the control molecular marker level of between X and Y indicates a normal molecular marker range; wherein a molecular marker level above Y indicates a susceptibility to liver disease in the human subject; and wherein a molecular marker level above Z indicates liver disease in the human subject. In additional embodiments, the present invention relates to an in vitro method for detecting increased molecular marker expression in a sample comprising: performing an in vitro nucleic acid detection assay on a sample wherein the nucleic acid detection assay comprises amplification of a nucleic acid molecule with at least a primer pair, said primer pair comprising a forward primer comprising a nucleotide sequence and a reverse primer comprising a nucleotide sequence that hybridizes to a molecular marker to produce amplified molecular marker nucleic acid wherein detecting an elevated level of a molecular marker in the sample when compared to a control molecular marker level indicates susceptibility to NAFLD, NASH, liver fibrosis or related disease of the liver.

The present invention also provides a method for monitoring liver treatment in a patient being treated with a modulator of a molecular target comprising: (a) performing a nucleic acid detection assay on a nucleic acid sample from the patient to detect the presence of a molecular marker in the patient's nucleic acid sample when compared to a reference molecular marker. level, wherein the nucleic acid detection assay comprises amplification of the molecular marker, and (b) determining the patient's responsiveness to treatment based on the level of the molecular marker, in the patient's nucleic acid sample, wherein the reference molecular marker. level of between X and Y indicates a normal molecular marker range and responsiveness to treatment; wherein a molecular marker level above Y indicates continuing susceptibility to liver disease and partial responsiveness to treatment in the patient; and wherein a molecular marker level above Z indicates liver disease in the patient and unresponsiveness to treatment. In certain embodiments, the molecular marker, is detected by hybridizing a labeled oligonucleotide probe to the amplified nucleic acid. In certain embodiments, the sample is a liver biopsy. In certain embodiments, the sample comprises hepatocytes. In certain embodiments, amplification of a nucleic acid comprises PCR or real time PGR (RT-PCR). In certain embodiments, the amplification of a nucleic acid comprises reverse transcriptase PCR. In certain embodiments, the forward and/or the reverse primer is detectably labeled. In certain embodiments, the method further comprises electrophoresis of the amplified nucleic acid. In certain embodiments, the method further comprises using a real-time PCR detection system. In certain embodiments, the control level represents the level of a molecular marker in liver of a healthy subject.

### Pharmaceutical Composition, Treatment And Administration:

The present invention provides a method of treating liver disease, including nonalcoholic steatohepatitis (NASH), nonalcoholic fatty liver disease (NAFLD), and/or liver fibrosis and related conditions in a subject in need thereof. The method includes administering to the subject an effective amount of a modulator of molecular target according to the present invention or its expression. In various embodiments, the method may further include measuring the expression or activity of the molecular target in a cell sample of the subject to be treated, and determining that molecular target activity or expression is increased or decreased after administration of the modulator, as compared to the level of molecular target activity or expression prior to administration of the modulator. Such a detected modulation confirms treatment of NASH, NAFLD, liver fibrosis and related conditions in the subject.

As further provided herein, a modulator useful in a method of the invention can be any type of molecule, for example, a polynucleotide, a peptide, a peptidomimetic, peptoids such as vinylogous peptoids, a small organic molecule, or the like, and can act in any of various ways to modulate the activity or expression of the molecular target(s). Further, the modulator can be administered in any way typical of an agent used to treat the particular type of liver disease or under conditions that facilitate contact of the modulator with the molecular target. Entry of a polynucleotide modulator into a cell, for example, can be facilitated by incorporating the polynucleotide into a viral vector that can infect the cells. If a viral vector specific for the cell type is not available, the vector can be modified to express a receptor (or ligand) specific for a ligand (or receptor) expressed on the target cell, or can be encapsulated within a liposome, which also can be modified to include such a ligand (or receptor). A peptide modulator can be introduced into a cell by various methods, including, for example, by engineering the peptide to contain a protein transduction domain such as the human immunodeficiency virus TAT protein transduction domain, which can facilitate translocation of the peptide into the cell.

An approach for therapy of liver disease and disorders is to express anti-sense constructs directed against the molecular target polynucleotides as described herein, and specifically administering them to liver cells, to inhibit gene function and prevent one or more of the symptoms and processes associated with the progression of NAFLD to NASH. Such treatment may also be useful in treating patients who already exhibit a progression to NASH, to reverse or alleviate one or more of the disease processes. Additionally, approaches utilizing one or more additional modulators are also expected to be useful for treating certain conditions. In certain instances, administering at least one additional therapeutic agent for treatment of liver disease may be useful.

Anti-sense constructs may be used to inhibit gene function to prevent progression of liver disease, e.g., to NASH. Antisense constructs, i.e., nucleic acid, such as RNA, constructs complementary to the sense nucleic acid or mRNA, are described in detail in U.S. Pat. No. 6, 100,090 (Monia et al.), and Neckers et al., 1992, Crit Rev Oncog 3(1-2): 175-231.

In a particular example, NAFLD and NASH may be treated or prevented by reducing the amount, expression or activity of molecular target in whole or in part in liver cells, for example by siRNAs capable of binding to and destroying the molecular target mRNA. Examples of such anti-molecular target modulators are provided herein, which function to downregulate the molecular target by RNA interference. The anti-molecular target modulator may comprise a Small Interfering RNA (siRNA) or Short Hairpin RNA (shRNA).

RNA interference (RNAi) is a method of post transcriptional gene silencing (PTGS) induced by the direct introduction of double-stranded RNA (dsRNA) and has emerged as a useful tool to knock out expression of specific genes in a variety of organisms. RNAi is described by Fire et al., Nature 391 :806-811 (1998). Other methods of PTGS are known and include, for example, introduction of a transgene or vims. Generally, in PTGS, the transcript of the silenced gene is synthesized but does not accumulate because it is rapidly degraded. Methods for PTGS, including RNAi are described, for example, in the Ambion.com world wide web site, in the directory “/hottopics/”, in the “rnai” file.

Suitable methods for RNAi in vitro are described herein. One such method involves the introduction of siRNA (small interfering RNA). Current models indicate that these 21-23 nucleotide dsRNAs can induce PTGS. Methods for designing effective siR As are described, for example, in the Ambion web site described above. RNA precursors such as Short Hairpin R As (shRNAs) can also be encoded by all or a part of the molecular target nucleic acid sequence.

Alternatively, double-stranded (ds) RNA is a powerful way of interfering with gene expression in a range of organisms that has recently been shown to be successful in mammals (Wianny and Zernicka-Goetz, 2000, Nat Cell Biol 2:70-75). Double stranded RNA corresponding to the sequence of a molecular target polynucleotide can be introduced into or expressed in oocytes and cells of a candidate organism to interfere with molecular target activity.

Other methods of modulating gene expression of the molecular target are known to those skilled in the art and include dominant negative approaches. An example of this approach, which could be utilized in the context of inhibiting, preventing, or treating NAFLD, NASH, liver fibrosis and related conditions is utilizing a molecular target mutant to block molecular target interaction or a small molecule chemical or mimetic which can block molecular target interaction. (Zhang H, et al., J Biol Chem. 2009 May 15; 284(20): 13355-62).

Molecular target gene expression may also be modulated by introducing peptides or small molecules which inhibit gene expression or functional activity. Thus, compounds identified by the assays described herein as binding to or modulating, such as down-regulating, the amount, activity or expression of a molecular target polypeptide may be administered to liver cells to prevent the function of molecular target polypeptide. Such a compound may be administered along with a pharmaceutically acceptable carrier in an amount effective to down-regulate expression or activity a molecular target. or by activating or down regulating a second signal, which controls molecular target expression, activity or amount, and thereby alleviating the abnormal condition.

Alternatively, gene therapy may be employed to control the endogenous production of a molecular target by the relevant cells such as liver cells in the subject. For example, a polynucleotide encoding a molecular target siRNA or a portion of this may be engineered for expression in a replication defective retroviral vector, as discussed below. The retroviral expression construct may then be isolated and introduced into a packaging cell transduced with a retroviral plasmid vector containing RNA encoding an anti-molecular target siRNA such that the packaging cell now produces infectious viral particles containing the sequence of interest. These producer ceils may be administered to a subject for engineering cells in vivo and regulating expression of the molecular target polypeptide in vivo. For overview of gene therapy, see Chapter 20, Gene Therapy and other Molecular Genetic-based Therapeutic Approaches, (and references cited therein) in Human Molecular Genetics, T Strachan and A P Read, BIOS Scientific Publishers Ltd (1996).

In some embodiments, the level of a molecular target is decreased in a liver cell. Furthermore, in such embodiments, treatment may be targeted to, or specific to, liver cells. The expression of a molecular marker may be specifically decreased only in diseased liver cells (i.e., those cells which are predisposed to the liver condition, or exhibiting liver disease already), and not substantially in other non-diseased liver cells. In these methods, expression of the molecular target may not be substantially reduced in other cells, i.e., cells which are not liver cells. Thus, in such embodiments, the level of molecular marker remains substantially the same or similar in non-liver cells in the course of or following treatment.

Liver cell specific reduction of molecular marker levels may be achieved by targeted administration, i.e., applying the treatment only to the liver cells and not other cells. However, in other embodiments, down-regulation of a molecular target expression in liver cells (and not substantially in other cell or tissue types) is employed. Such methods may advantageously make use of liver specific expression vectors, for liver specific expression of for example siRNAs, as described in further detail below.

To prepare pharmaceutical or sterile compositions of the compositions of the present invention, the modulators, or similar compositions may be admixed with a pharmaceutically acceptable carrier or excipient. See, e.g., Remington's Pharmaceutical Sciences and U.S. Pharmacopeia: National Formulary, Mack Publishing Company, Easton, Pa. (1984).

Formulations of therapeutic and diagnostic modulators may be prepared by mixing with acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Deliker, N.Y.; Lieberman, et al. (eds.) (1990) Pharmaceutical Dosage Forms: Tablets, Marcel Dekker, N.Y.; Lieberman, el al. (eds.) (1990) Pharmaceutical Dosage Forms: Disperse Systems, Marcel Dekker, N.Y.; Weiner and Kotkoskie (2000) Excipient Toxicity and Safety. Marcel Dekker, Inc., New York, N.Y.).

Toxicity and therapeutic efficacy of the modulators of the present invention, administered alone or in combination with another agent, can be determined by standard pharmaceutical procedures in cell cultures or experimental animals, e.g., for determining the LD (the dose lethal to 50% of the population) and the D (the dose therapeutically effective in 50% of the population). The dose ratio between toxic and therapeutic effects is the therapeutic index (LD50 ED50). In particular aspects, therapeutic compositions exhibiting high therapeutic indices are desirable. The data obtained from these cell culture assays and animal studies can be used in formulating a range of dosage for use in human. The dosage of such compounds lies preferably within a range of circulating concentrations that include the ED50 with little or no toxicity. The dosage may vary within this range depending upon the dosage form employed and the route of administration.

In an embodiment of the invention, a composition of the invention is administered to a subject in accordance with the Physicians' Desk Reference 2003 (Thomson Healthcare; 57th edition (Nov. 1, 2002)).

The mode of administration can vary. Suitable routes of administration include oral, rectal, transmucosal, intestinal, parenteral; intramuscular, subcutaneous, intradermal, intramedullary, intrathecal, direct intraventricular, intravenous intraperitoneal, intranasal, intraocular, inhalation, insufflation, topical, cutaneous, transdermal, or intra-arterial.

In particular embodiments, the composition or therapeutic can be administered by an invasive route such as by injection (see above). In further embodiments of the invention, the composition, therapeutic, or pharmaceutical composition thereof, is administered intravenously, subcutaneously, intramuscularly, intraarterially, intra-particularly (e.g. in arthritis joints), intratumorally, or by inhalation, aerosol delivery. Administration by noninvasive routes (e.g., orally; for example, in a pill, capsule or tablet) is also within the scope of the present invention.

Compositions can be administered with medical devices known in the art. For example, a pharmaceutical composition of the invention can be administered by injection with a hypodermic needle, including, e.g., a prefilled syringe or autoinjector.

The pharmaceutical compositions of the invention may also be administered with a needleless hypodermic injection device; such as the devices disclosed in U.S. Pat. Nos. 6,620,135; 6,096,002: 5,399,163; 5,383,851; 5,312,335; 5,064,413; 4,941,880; 4,790,824 or 4,596,556.

Alternately, one may administer the modulators (inhibitors and activators) or related compounds in a local rather than systemic manner, for example, via injection of directly into the desired target site, often in a depot or sustained release formulation. Furthermore, one may administer the composition in a targeted drug delivery system, for example, in a liposome coated with a tissue-specific antibody, targeting, for example, the liver, and more specifically hepatocytes. The liposomes will be targeted to and taken up selectively by the desired tissue. Also included in a targeted drug delivery system is nanoparticle specific liver delivery of the modulators, alone or in combination with other similar activators or inhibitors. A summary of various delivery methods and techniques of siRNA administration in ongoing clinical trials is provided in Zuckerman and Davis 2015; Nature Rev. Drug Discovery, Vol. 14: 843-856, Dec, 2015.

Any of the modulators described herein, or any combination thereof, can also comprise a delivery vehicle, including liposomes, for administration to a subject, carriers and diluents and their salts, and/or can be present in pharmaceutically acceptable formulations. For example methods for the delivery of nucleic acid molecules are described in Akhtar et al., 1992, Trends Cell Bio., 2, 139; DELIVERY STRATEGIES FOR ANTISENSE OLIGONUCLEOTIDE THERAPEUTICS, ed. Akbtar, 1995, Maurer et al., 1999, Mol. Membr. Biol, 16, 129-140: Holland and Huang, 1999, Handb. Exp. Pharmacol, 137, 165-192; and Lee et al, 2000, ACS Symp. Ser., 752, 184-192. U.S. Pat. No. 6,395,713 and PCT Publication No. WO 94/02595 further describe the general methods for delivery of nucleic acid molecules. These protocols can be utilized for the delivery of virtually any nucleic acid molecule.

Any of the therapeutics described herein including, or any combination thereof can also be administered to a desired target by a variety of methods known to those of skill in the art, including, but not restricted to, encapsulation in liposomes, by iontophoresis, or by incorporation into other vehicles, such as hydrogels, cyclodextrins, biodegradable nanocapsules, and bioadhesive microspheres, or by proteinaceous vectors (see PCT Publication No. WO 00/53722). Alternatively, the therapeutic/vehicle combination is locally delivered by direct injection or by use of an infusion pump. Direct injection of the composition, whether subcutaneous, intramuscular, or intradermal, can take place using standard needle and syringe methodologies, or by needle- free technologies such as those described in Conry et al., 1999, Clin. Cancer Res., 5, 2330-2337 and PCT Publication No. WO 99/3 1262.

The administration regimen depends on several factors, including the serum or tissue turnover rate of the therapeutic composition, the level of symptoms, and the accessibility of the target cells in the biological matrix. Preferably, the administration regimen delivers sufficient therapeutic composition to effect improvement in the target disease state, while simultaneously minimizing undesired side effects. Accordingly, the amount of biologic delivered depends in part on the particular therapeutic composition and the severity of the condition being treated.

Determination of the appropriate dose is made by the clinician e.g., using parameters or factors known or suspected in the art to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. In general, it is desirable that a biologic that will be used is derived from the same species as the animal targeted for treatment, thereby minimizing any immune response to the reagent.

As used herein, “activate”, “inhibit”, “increase”, “antagonize”, “agonize” or “treat” or “treatment” includes a postponement of development of the symptoms associated with a disorder and/or a reduction in the severity of the symptoms of such disorder. The terms further include ameliorating existing uncontrolled or unwanted symptoms, preventing additional symptoms, and ameliorating or preventing the underlying causes of such symptoms. Thus, the terms denote that a beneficial result has been conferred on a vertebrate subject with a disorder, disease or symptom or with the potential to develop such a disorder, disease or symptom.

As used herein, the terms “therapeutically effective amount”, “therapeutically effective dose” and “effective amount” refer to an amount of a modulator or the present invention that, when administered alone or in combination with an additional therapeutic agent to a cell, tissue, or patient, is effective to cause a measurable improvement in one or more symptoms of a disease or condition or the progression of such disease or condition. A therapeutically effective dose further refers to that amount of the compound sufficient to result in at least partial amelioration of symptoms, e.g., treatment, healing, prevention or amelioration of the relevant medical condition, or an increase in rate of treatment, healing, prevention or amelioration of such conditions. When applied to an individual active ingredient administered alone a therapeutically effective dose refers to that ingredient alone. When applied to a combination, a therapeutically effective dose refers to combined amounts of the active ingredients that result in the therapeutic effect, whether administered in combination, serially or simultaneously. An effective amount of a therapeutic will result in an improvement of a diagnostic measure or parameter by at least 10%; usually by at least 20%; preferably at least about 30%; more preferably at least 40%, and most preferably by at least 50%. An effective amount can also result in an improvement in a subjective measure in cases where subjective measures are used to assess disease severity.

### General Methods:

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, N.Y.; Sambrook and Russell (2001) Molecular Cloning, 3^{rd} eel., Cold Spring Harbor Laboratory Press. Cold Spring Harbor, N.Y.; Wu (1993) Recombinant DMA, Vol. 217, Academic Press, San Diego, Calif.). Standard methods also appear in Ausbei, et a (2001) Current Protocols in Molecular Biology, Vols-4 , John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan. et a (2000) Current Protocols in Protein Science, Vol. J, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, N.Y., pp. 16.0.5-16.22.17; Sigma-Aldrich, Co. (2001) P oduc s for Life Science Research, St. Louis, Mo.; pp. 45-89; Amersham Pharmacia Biotech (2001) BioDireclory. Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra). Standard techniques for characterizing ligand/receptor interactions are available (see, e.g., Coligan, et al. (2001) Current Protocols in Immunology, Vol. 4, John Wiley, Inc., New York).

### EXAMPLES

### Example 1

### Tissue Generation and Single Nuclei RNA Sequencing

Bioprinted tissues comprised of cells isolated from the livers of either healthy or NAFLD donors were generated as previously described (references: U.S. Pat. Nos. 9,442,105 and 10,400,219), using either human umbilical vein endothelial cells (HUVECs) or liver endothelial cells (LECs) as the endothelial cell source. Tissues were harvested and snap frozen at various timepoints over a 14-day time course. Snap frozen tissues were subjected to mechanical and chemical disruption to isolate intact nuclei, which were further enriched by fluorescence-activated cell sorting (FACS) based methodology using propidium iodide staining for positive selection. Nuclei were immediately processed on the 10× Genomics Chromium Controller using their Single Cell 3′ V3 reagents. RNA from the captured and barcoded nuclei was then converted to cDNA, amplified to generate libraries and subjected to sequencing on an IIlumina NovaSeq 6000 Sequencing System.

The proportion of nuclei expressing a given gene was determined for each 3-day old healthy and disease bioprinted tissue and compared. Each healthy and disease group consisted of duplicate tissues. As summarized in table 2, CHRM, DMKN, QPRT and SLC12A8 all demonstrated upregulation of gene expression at this early timepoint in disease bioprinted tissues relative to healthy tissues. Note that in the case of QPRT, the healthy tissues contained HUVECs.

TABLE 2
Differentially regulated genes in disease derived 3D bioprinted tissues.

| | Log₂ Fold | |
|---|---|---|
| | Change In | Fold Change In |
| TARGET Name | Expression | Expression |
| CHRM2 | 1.12 | 2.17 |
| DMKN | 0.56 | 1.47 |
| QPRT | 1.18 | 2.27 |
| SLC12A8 | 0.91 | 1.88 |

Bulk RNA sequencing of four cell types isolated from human livers demonstrated that CHRM2, DMKN, LOXL4, QPRT and SLC12A8 were upregulated in endothelial cells derived from the liver of NAFLD donors relative to those without the disease. Consistent with these data, single nuclei RNA sequencing of bioprinted tissues, which include liver endothelial cells, demonstrated upregulation of CHRM2, DMKN, QPRT and SLC12A8 in tissues generated from disease donor cells. Notably, this differential gene expression occurred in bioprinted tissues comprised of cells from donors distinct from those analyzed by bulk RNA sequencing.

Bioprinted tissues generated from cells of NAFLD donors maintain cues that drive fibrosis as evident by the higher amount of collagen deposition in the disease derived tissues, which occurs within the first week of culturing. Because CHRM2, DMKN, QPRT and SLC12A8 are upregulated in both the isolated liver endothelial cells and at the early stage of the bioprinted tissues, and this upregulation is observed across different NAFLD donors, these genes are clinically relevant novel drivers of liver disease, including NAFLD, NASH, fibrosis of the liver and related conditions.

Although only a few embodiments have been disclosed in detail above in this specification. The specification describes specific examples to accomplish a more general goal that may be accomplished in another way. This disclosure is intended to be exemplary, and the claims are intended to cover any modification or alternative which might be predictable to a person having ordinary skill in the art.

Having illustrated and described the principles of the invention in exemplary embodiments, it should be apparent to those skilled in the art that the described examples are illustrative embodiments and can be modified in arrangement and detail without departing from such principles. Techniques from any of the examples can be incorporated into one or more of any of the other examples. It is intended that the specification and examples be considered as exemplary only, with a true scope and spirit of the invention being indicated by the following claims.

We claim:

1. A molecular marker of liver disease, said molecular marker being selected from the group consisting of at least one of LOXL4, CHRM2, DMKN, QPRT, and SLC12A8.

2. The molecular marker of claim 1 further comprising a molecular target useful for screening for modulators of liver disease in a patient wherein said molecular target is selected from the group consisting of at least one of LOXL4, CHRM2, DMKN, QPRT and SLC12A8.

3. The molecular marker of claim 1 further comprising a molecular target capable of treating liver disease in a patient when contacted by a modulator, said molecular target being selected from the group consisting of at least one of LOXL4, CHRM2, DMKN, QPRT, and SLC12A8.

4. The molecular marker of claim 2 wherein the molecular target is QPRT.

5. The molecular marker of claim 3 wherein the molecular target is QPRT.

6. The molecular marker of claim 1 wherein the expression level of the molecular marker is greater in a patient with liver disease. The molecular marker of claim 6 wherein the liver disease in said patient is selected from the group consisting of non-alcoholic fatty liver disease, nonalcoholic steatohepatitis, fibrosis of the liver and combinations thereof.

8. A method of identifying modulators for treating diseases of the liver in a patient, the method comprising:
a. Providing one or more molecular targets associated with liver disease;
b. Contacting the one or more molecular targets with one or more potential modulators; and
c. Selecting those modulators that regulate the expression and/or activity of the one or more molecular targets, wherein the molecular target is selected from the group consisting of at least one of LOXL4, CHRM2, DMKN, QPRT, and SLC12A8.

9. The method of claim 8 wherein the one or more modulators are natural or synthetic modulators.

10. The method of claim 9 wherein the natural or synthetic modulator is selected from the group consisting of cytokines, cytokine variants, analogues, muteins, antibodies, binding compounds derived from antibodies, small molecules, peptide mimetics, siRNA, nucleic acids, proteins or an extract made from biological materials such as bacteria, plants, fungi, or animal cells or tissues.

11. The method claim 8 further comprising preventing or treating liver disease in a patient by administering to said patient an effective amount of one or more modulators, said one or more modulators modulating the expression and/or activity of one or more molecular targets selected from the group consisting of at least one of LOXL4, CHRM2, DMKN, QPRT or SLC12A8.

12. The method of claim 11 wherein the modulator inhibits the expression and/or activity of at least one or more of LOXL4, CHRM2, DMKN, QPRT and/or SLC12A8.

13. The method of claim 11 wherein the modulator increases the expression and/or activity of at least one of LOXL4, CHRM2, DMKN, QPRT and/or SLC12A8.

14. The method of claim 13 wherein the modulator is activator of QPRT expression and/or activity.

15. The method of claim 14 wherein the activator is selected from the group consisting of cytokines, cytokine variants, analogues, muteins, antibodies, binding compounds derived from antibodies, small molecules, peptide mimetics, siRNA, nucleic acids, proteins or an extract made from biological materials such as bacteria, plants, fungi, or animal cells or tissues.

16. The method of claim 10 wherein the liver disease is selected from the group consisting of non-alcoholic fatty liver disease, nonalcoholic steatohepatitis, fibrosis of the liver and combinations thereof.

17. A method of determining whether a subject has liver disease comprising:
a. providing a molecular marker panel of two or more molecular markers comprising at least one molecular marker selected from group consisting of LOXL4, CHRM, DMKN, QRPT and SLB12AB;
b. detecting the level of expression of the molecular markers in the panel in a sample from a patient to give molecular marker values that correspond to the molecular markers in the molecular marker panel and that are higher than a control level of the at least one respective molecular marker in the molecular marker panel to determine whether the subject has or has a predisposition for liver disease.

18. The method of claim 17 wherein each molecular marker is expressed as a protein molecular maker selected from group consisting of LOXL4, CHRM, DMKN, QRPT and SLB12AB.

19. The method of claim 17 wherein the liver disease is selected from the group consisting of non-alcoholic fatty liver disease, nonalcoholic steatohepatitis, fibrosis of the liver and combinations thereof.

20. The method of claim 17 wherein the determining of whether a patient has liver disease includes the early diagnosing of liver disease, determining the predisposition of the patient to develop liver disease, determining the severity of liver disease in the patient and/or monitoring the effect of therapeutic administered to the patient.


SEQUENCE LISTING (21 sequences)

SEQ ID NO: 1 - protein, 756 aa, Artificial Sequence
  LOXL4 Protein
MAWSPPATLFLFLLLLGQPPPSRPQSLGTTKLRLVGPESKPEEGRLEVLHQGQWGTVCDDNFAIQEATVACRQLGFEAALTWAHSAKYGQGEGPIWLDNVRCVGTESSLDQCGSNGWGVSDCSHSEDVGVICHPRRHRGYLSETVSNALGPQGRRLEEVRLKPILASAKQHSPVTEGAVEVKYEGHWRQVCDQGWTMNNSRVVCGMLGFPSEVPVDSHYYRKVWDLKMRDPKSRLKSLTNKNSFWIHQVTCLGTEPHMANCQVQVAPARGKLRPACPGGMHAVVSCVAGPHFRPPKTKPQRKGSWAEEPRVRLRSGAQVGEGRVEVLMNRQWGTVCDHRWNLISASVVCRQLGFGSAREALFGARLGQGLGPIHLSEVRCRGYERTLSDCPALEGSQNGCQHENDAAVRCNVPNMGFQNQVRLAGGRIPEEGLLEVQVEVNGVPRWGSVCSENWGLTEAMVACRQLGLGFAIHAYKETWFWSGTPRAQEVVMSGVRCSGTELALQQCQRHGPVHCSHGGGRFLAGVSCMDSAPDLVMNAQLVQETAYLEDRPLSQLYCAHEENCLSKSADHMDWPYGYRRLLRFSTQIYNLGRTDFRPKTGRDSWVWHQCHRHYHSIEVFTHYDLLTLNGSKVAEGHKASFCLEDTNCPTGLQRRYACANFGEQGVTVGCWDTYRHDIDCQWVDITDVGPGNYIFQVIVNPHYEVAESDFSNNMLQCRCKYDGHRVWLHNCHTGNSYPANAELSLEQEQRLRNNLI

SEQ ID NO: 2 - protein, 466 aa, Artificial Sequence
  CHRM2 Protein
MNNSTNSSNNSLALTSPYKTFEVVFIVLVAGSLSLVTIIGNILVMVSIKVNRHLQTVNNYFLFSLACADLIIGVFSMNLYTLYTVIGYWPLGPVVCDLWLALDYVVSNASVMNLLIISFDRYFCVTKPLTYPVKRTTKMAGMMIAAAWVLSFILWAPAILFWQFIVGVRTVEDGECYIQFFSNAAVTFGTAIAAFYLPVIIMTVLYWHISRASKSRIKKDKKEPVANQDPVSPSLVQGRIVKPNNNNMPSSDDGLEHNKIQNGKAPRDPVTENCVQGEEKESSNDSTSVSAVASNMRDDEITQDENTVSTSLGHSKDENSKQTCIRIGTKTPKSDSCTPTNTTVEVVGSSGQNGDEKQNIVARKIVKMTKQPAKKKPPPSREKKVTRTILAILLAFIITWAPYNVMVLINTFCAPCIPNTVWTIGYWLCYINSTINPACYALCNATFKKTFKHLLMCHYKNIGATR

SEQ ID NO: 3 - protein, 90 aa, Artificial Sequence
  DMKN Protein [Transcript  1]
MNMKPATASALLLLLLGLAWTQGSHGWGADASSLQKRAGRDDQNYNYNQHAYPTAYGGKYSVKTPAKGGVSPSSSASRVQPGLLQWVKFW

SEQ ID NO: 4 - protein, 476 aa, Artificial Sequence
  DMKN Protein [Transcript  2]
MKFQGPLACLLLALCLGSGEAGPLQSGEESTGTNIGEALGHGLGDALSEGVGKAIGKEAGGAAGSKVSEALGQGTREAVGTGVRQVPGFGVADALGNRVGEAAHALGNTGHEIGRQAEDVIRHGADAVRGSWQGVPGHNGAWETSGGHGIFGSQGGLGGQGQGNPGGLGTPWVHGYPGNSAGSFGMNPQGAPWGQGGNGGPPNFGTNTQGAVAQPGYGSVRASNQNEGCTNPPPSGSGGGSSNSGGGSGSQSGSSGSGSNGDNNNGSSSGGSSSGSSSGGSSGGSSGGSSGNSGGSRGDSGSESSWGSSTGSSSGNHGGSGGGNGHKPGCEKPGNEARGSGESGIQNSETSPGMFNFDTFWKNFKSKLGFINWDAINKNQVPPPSTRALLYFSRLWEDFKQNTPFLNWKAIIEGADASSLQKRAGRDDQNYNYNQHAYPTAYGGKYSVKTPAKGGVSPSSSASRVQPGLLQWVKFW

SEQ ID NO: 5 - protein, 465 aa, Artificial Sequence
  DMKN Protein [Transcript  3]
MKFQGPLACLLLALCLGSGEAGPLQSGEESTGTNIGEALGHGLGDALSEGVGKAIGKEAGGAAGSKVSEALGQGTREAVGTGVRQVPGFGVADALGNRVGEAAHALGNTGHEIGRQAEDVIRHGADAVRGSWQGVPGHNGAWETSGGHGIFGSQGGLGGQGQGNPGGLGTPWVHGYPGNSAGSFGMNPQGAPWGQGGNGGPPNFGTNTQGAVAQPGYGSVRASNQNEGCTNPPPSGSGGGSSNSGGSSTGSSSGNHGGSGGGNGHKPGCEKPGNEARGSGESGIQGFRGQGVSSNMREISKEGNRLLGGSGDNYRGQGSSWGSGGGDAVGGVNTVNSETSPGMFNFDTFWKNFKSKLGFINWDAINKNQVPPPSTRALLYFSRLWEDFKQNTPFLNWKAIIEGADASSLQKRAGRDDQNYNYNQHAYPTAYGGKYSVKTPAKGGVSPSSSASRVQPGLLQWVKFW

SEQ ID NO: 6 - protein, 386 aa, Artificial Sequence
  DMKN Protein [Transcript  4]
MKFQGPLACLLLALCLGSGEAGPLQSGEESTGTNIGEALGHGLGDALSEGVGKAIGKEAGGAAGSKVSEALGQGTREAVGTGVRQVPGFGVADALGNRVGEAAHALGNTGHEIGRQAEDVIRHGADAVRGSWQGVPGHNGAWETSGGHGIFGSQGGLGGQGQGNPGGLGTPWVHGYPGNSAGSFGMNPQGAPWGQGGNGGPPNFGTNTQGAVAQPGYGSVRASNQNEGCTNPPPSGSGGGSSNSGGGSGSQSGSSGSGSNGDNNNGSSSGGSSSGSSSGGSSGGSSGGSSGNSGGSRGDSGSESSWGSSTGSSSGNHGGSGGGNGHKPGCEKPGNEARGSGESGIQNSETSPGMFNFDTFWKNFKSKLGFINWDAINKDQRSSRIP

SEQ ID NO: 7 - protein, 398 aa, Artificial Sequence
  DMKN Protein [Transcript  5]
MKFQGPLACLLLALCLGSGEAGPLQSGEESTGTNIGEALGHGLGDALSEGVGKAIGKEAGGAAGSKVSEALGQGTREAVGTGVRQVPGFGVADALGNRVGEAAHALGNTGHEIGRQAEDVIRHGADAVRGSWQGVPGHNGAWETSGGHGIFGSQGGLGGQGQGNPGGLGTPWVHGYPGNSAGSFGMNPQGAPWGQGGNGGPPNFGTNTQGAVAQPGYGSVRASNQNEGCTNPPPSGSGGGSSNSGGGSGSQSGSSGSGSNGDNNNGSSSGGSSSGSSSGGSSGGSSGGSSGNSGGSRGDSGSESSWGSSTGSSSGNHGGSGGGNGHKPGCEKPGNEARGSGESGIQGFRGQGVSSNMRNSETSPGMFNFDTFWKNFKSKLGFINWDAINKDQRSSRIP

SEQ ID NO: 8 - protein, 189 aa, Artificial Sequence
  DMKN Protein [Transcript  6]
MLGITSCSDQQAKEGEGLEGSSTGSSSGNHGGSGGGNGHKPGCEKPGNEARGSGESGIQNSETSPGMFNFDTFWKNFKSKLGFINWDAINKNQVPPPSTRALLYFSRLWEDFKQNTPFLNWKAIIEGADASSLQKRAGRDDQNYNYNQHAYPTAYGGKYSVKTPAKGGVSPSSSASRVQPGLLQWVKFW

SEQ ID NO: 9 - protein, 449 aa, Artificial Sequence
  DMKN Protein [Transcript  8]
MKFQGPLACLLLALCLGSGEAGPLQSGEESTGTNIGEALGHGLGDALSEGVGKAIGKEAGGAAGSKVSEALGQGTREAVGTGVRQVPGFGVADALGNRVGEAAHALGNTGHEIGRQAEDVIRHGADAVRGSWQGVPGHNGAWETSGGHGIFGSQGGLGGQGQGNPGGLGTPWVHGYPGNSAGSFGMNPQGAPWGQGGNGGPPNFGTNTQGAVAQPGYGSVRASNQNEGCTNPPPSGSGGGSSNSGGSSTGSSSGNHGGSGGGNGHKPGCEKPGNEARGSGESGIQGQGSSWGSGGGDAVGGVNTVNSETSPGMFNFDTFWKNFKSKLGFINWDAINKNQVPPPSTRALLYFSRLWEDFKQNTPFLNWKAIIEGADASSLQKRAGRDDQPGAGWQEVAAVTSKNYNYNQHAYPTAYGGKYSVKTPAKGGVSPSSSASRVQPGLLQWVKFW

SEQ ID NO: 10 - protein, 436 aa, Artificial Sequence
  DMKN Protein [Transcript  9]
MKFQGPLACLLLALCLGSGEAGPLQSGEESTGTNIGEALGHGLGDALSEGVGKAIGKEAGGAAGSKVSEALGQGTREAVGTGVRQVPGFGVADALGNRVGEAAHALGNTGHEIGRQAEDVIRHGADAVRGSWQGVPGHNGAWETSGGHGIFGSQGGLGGQGQGNPGGLGTPWVHGYPGNSAGSFGMNPQGAPWGQGGNGGPPNFGTNTQGAVAQPGYGSVRASNQNEGCTNPPPSGSGGGSSNSGGGSGSQSGSSGSGSNGDNNNGSSSGGSSSGSSSGGSSGGSSGGSSGNSGGSRGDSGSESSWGSSTGSSSGNHGGSGGGNGHKPGCEKPGNEARGSGESGIQGFRGQGVSSNMREISKEGNRLLGGSGDNYRGQGSSWGSGGGDAVGGVNTVNSETSPGMFNFDTFWKNFKSKLGFINWDAINKDQRSSRIP

SEQ ID NO: 11 - protein, 369 aa, Artificial Sequence
  DMKN Protein [Transcript  10]
MKFQGPLACLLLALCLGSGEAGPLQSGEESTGTNIGEALGHGLGDALSEGVGKAIGKEAGGAAGSKVSEALGQGTREAVGTGVRQVPGFGVADALGNRVGEAAHALGNTGHEIGRQAEDVIRHGADAVRGSWQGVPGHNGAWETSGGHGIFGSQGGLGGQGQGNPGGLGTPWVHGYPGNSAGSFGMNPQGAPWGQGGNGGPPNFGTNTQGAVAQPGYGSVRASNQNEGCTNPPPSGSGGGSSNSGGGSGSQSGSSGSGSNGDNNNGSSSGGSSSGSSSGGSSGGSSGGSSGNSGGSRGDSGSESSWGSSTGSSSGNHGGSGGGNGHKPGNSETSPGMFNFDTFWKNFKSKLGFINWDAINKDQRSSRIP

SEQ ID NO: 12 - protein, 203 aa, Artificial Sequence
  DMKN Protein [Transcript  11]
MLGITSCSDQQAKEGEGLEGSSTGSSSGNHGGSGGGNGHKPGCEKPGNEARGSGESGIQNSETSPGMFNFDTFWKNFKSKLGFINWDAINKNQVPPPSTRALLYFSRLWEDFKQNTPFLNWKAIIEGADASSLQKRAGRDDQPGAGWQEVAAVTSKNYNYNQHAYPTAYGGKYSVKTPAKGGVSPSSSASRVQPGLLQWVKFW

SEQ ID NO: 13 - protein, 172 aa, Artificial Sequence
  DMKN Protein [Transcript  12]
MLGITSCSDQQAKEGEGLEGSSTGSSSGNHGGSGGGNGHKPGNSETSPGMFNFDTFWKNFKSKLGFINWDAINKNQVPPPSTRALLYFSRLWEDFKQNTPFLNWKAIIEGADASSLQKRAGRDDQNYNYNQHAYPTAYGGKYSVKTPAKGGVSPSSSASRVQPGLLQWVKFW

SEQ ID NO: 14 - protein, 186 aa, Artificial Sequence
  DMKN Protein [Transcript  13]
MLGITSCSDQQAKEGEGLEGSSTGSSSGNHGGSGGGNGHKPGNSETSPGMFNFDTFWKNFKSKLGFINWDAINKNQVPPPSTRALLYFSRLWEDFKQNTPFLNWKAIIEGADASSLQKRAGRDDQPGAGWQEVAAVTSKNYNYNQHAYPTAYGGKYSVKTPAKGGVSPSSSASRVQPGLLQWVKFW

SEQ ID NO: 15 - protein, 123 aa, Artificial Sequence
  DMKN Protein [Transcript  14]
MFNFDTFWKNFKSKLGFINWDAINKNQVPPPSTRALLYFSRLWEDFKQNTPFLNWKAIIEGADASSLQKRAGRDDQNYNYNQHAYPTAYGGKYSVKTPAKGGVSPSSSASRVQPGLLQWVKFW

SEQ ID NO: 16 - protein, 99 aa, Artificial Sequence
  DMKN Protein [Transcript  16]
MLGITSCSDQQAKEGEGLEGSSTGSSSGNHGGSGGGNGHKPGCEKPGNEARGSGESGIQNSETSPGMFNFDTFWKNFKSKLGFINWDAINKDQRSSRIP

SEQ ID NO: 17 - protein, 82 aa, Artificial Sequence
  DMKN Protein [Transcript  17]
MLGITSCSDQQAKEGEGLEGSSTGSSSGNHGGSGGGNGHKPGNSETSPGMFNFDTFWKNFKSKLGFINWDAINKDQRSSRIP

SEQ ID NO: 18 - protein, 297 aa, Artificial Sequence
  QPRT Protein [Transcript  1]
MDAEGLALLLPPVTLAALVDSWLREDCPGLNYAALVSGAGPSQAALWAKSPGVLAGQPFFDAIFTQLNCQVSWFLPEGSKLVPVARVAEVRGPAHCLLLGERVALNTLARCSGIASAAAAAVEAARGAGWTGHVAGTRKTTPGFRLVEKYGLLVGGAASHRYDLGGLVMVKDNHVVAAGGVEKAVRAARQAADFALKVEVECSSLQEAVQAAEAGADLVLLDNFKPEELHPTATVLKAQFPSVAVEASGGITLDNLPQFCGPHIDVISMGMLTQAAPALDFSLKLFAKEVAPVPKIH

SEQ ID NO: 19 - protein, 152 aa, Artificial Sequence
  QPRT Protein [Transcript  2]
MGPPSLHSPTPSLGPLGALVLSSQHTSPDSCKSPWTLKAVRAARQAADFALKVEVECSSLQEAVQAAEAGADLVLLDNFKPEELHPTATVLKAQFPSVAVEASGGITLDNLPQFCGPHIDVISMGMLTQAAPALDFSLKLFAKEVAPVPKIH

SEQ ID NO: 20 - protein, 149 aa, Artificial sequence
  QPRT Protein [Transcript  3]
MDAEGLALLLPPVTLAALVDSWLREDCPGLNYAALAVRAARQAADFALKVEVECSSLQEAVQAAEAGADLVLLDNFKPEELHPTATVLKAQFPSVAVEASGGITLDNLPQFCGPHIDVISMGMLTQAAPALDFSLKLFAKEVAPVPKIH

SEQ ID NO: 21 - protein, 714 aa, Artificial Sequence
  SLC12A8 Protein
MTQMSQVQELFHEAAQQDALAQPQPWWKTQLFMWEPVLFGTWDGVFTSCMINIFGVVLFLRTGWLVGNTGVLLGMFLVSFVILVALVTVLSGIGVGERSSIGSGGVYSMISSVLGGQTGGTIGLLYVFGQCVAGAMYITGFAESISDLLGLGNIWAVRGISVAVLLALLGINLAGVKWIIRLQLLLLFLLAVSTLDFVVGSFTHLDPEHGFIGYSPELLQNNTLPDYSPGESFFTVFGVFFPAATGVMAGFNMGGDLREPAASIPLGSLAAVGISWFLYIVFVFLLGAICTREALRYDFLIAEKVSLMGFLFLLGLYISSLASCMGGLYGAPRILQCIAQEKVIPALACLGQGKGPNKTPVAAICLTSLVTMAFVFVGQVNVLAPIVTINFMLTYVAVDYSYFSLSMCSCSLTPVPEPVLREGAEGLHCSEHLLLEKAPSYGSEGPAQRVLEGTLLEFTKDMDQLLQLTRKLESSQPRQGEGNRTPESQKRKSKKATKQTLQDSFLLDLKSPPSFPVEISDRLPAASWEGQESCWNKQTSKSEGTQPEGTYGEQLVPELCNQSESSGEDFFLKSRLQEQDVWRRSTSFYTHMCNPWVSLLGAVGSLLIMFVIQWVYTLVNMGVAAIVYFYIGRASPGLHLGSASNFSFFRWMRSLLLPSCRSLRSPQEQIILAPSLAKVDMEMTQLTQENADFATRDRYHHSSLVNREQLMPHY